(19) United States
(12) Patent Application Publication
(10) Pub. No.: US 2025/0318740 A1
(43) Pub. Date: Oct. 16, 2025

(54) IMPLANTABLE SENSING DEVICE TO MEASURE BLOOD PRESSURE

(71) Applicant: Qura, Inc., Duxbury, MA (US)

(72) Inventors: William G. Hendren, Duxbury, MA (US); Jean-Noel Fehr, Neuchatel (CH); Stefan Bauer, Bern (CH); Dieter Ulrich, Duxbury, MA (US); Dominik Täschler, Bern (CH)

(73) Assignee: Qura, Inc., Duxbury, MA (US)

(21) Appl. No.: 19/191,830

(22) Filed: Apr. 28, 2025

Related U.S. Application Data
(63) Continuation of application No. PCT/US2023/036409, filed on Oct. 31, 2023.
(60) Provisional application No. 63/381,616, filed on Oct. 31, 2022.

(51) Int. Cl. A61B 5/0215; A61B 5/00; A61B 5/11

(52) U.S. Cl. CPC A61B 5/0215; A61B 5/1126; A61B 5/6847; A61B 2560/0219; A61B 2560/0223; A61B 2562/0219; A61B 2562/0247; A61B 2562/164

(57) ABSTRACT

An implantable pressure-sensing device can be used to measure blood pressure. It can include a substrate to secure the implantable pressure-sensing device within 0 mm to 10 mm from a blood vessel, a pressure-sensing element projecting from an outer surface of the substrate and configured to sense blood pressure within the blood vessel, circuitry disposed on the substrate and in electrical communication with the pressure-sensing element and configured to receive data from the pressure-sensing element, and a power management system. The pressure-sensing device may also include a structure disposed around the pressure-sensing element configured to transmit pressure waves from the blood vessel towards the pressure-sensing element. The pressure-sensing device may also include a multilayer ceramic/polymer coating.

### CROSS REFERENCE TO RELATED APPLICATION(S)

The present application is a bypass continuation of International Application No. PCT/US2023/036409, filed Oct. 31, 2023, which claims the priority benefit, under 35 U.S.C. 119 (e), of U.S. Application No. 63/381,616, filed Oct. 31, 2022, which is incorporated herein by reference in its entirety for all purposes.

### BACKGROUND

Blood pressure is one of the core physiological measurements of interest in virtually all healthcare contexts because it provides insight into a patient's cardiac function, volume status, organ perfusion, and overall hemodynamic stability. High blood pressure, or hypertension, is an immense global health care problem that affects billions of people, with two-thirds of them living in middle- to low-income countries. Hypertension significantly increases the risk of developing cardiovascular disease and renal disease, and of having a heart attack or stroke, among other life-threatening conditions. In the United States, hypertension affects nearly one in two adults and despite the common nature of the condition, only about 24% have their blood pressure controlled. The United States Surgeon General has recently made hypertension control a national priority supported by The Surgeon General's Call to Action to Control Hypertension.

Blood pressure is the measurement of the pressure or force of blood pushing against blood vessel walls. The heart pumps blood into the arteries which carry oxygenated blood throughout the body. Blood pressure can be measured in any artery but there are differences in measured pressures related to the size, location, and intrinsic structure of blood vessel walls.

In current clinical practice, blood pressure is typically monitored using a noninvasive sphygmomanometer, otherwise known as a blood pressure cuff, usually over the brachial artery. This practice has changed little in over a century because of its ease of use. Despite its relative ease of use, there are significant problems related to cuff measurements which can lead to errors that inappropriately alter health management decisions in about 20% to about 45% of cases. In high risk surgical or intensive care unit (ICU) patients, blood pressure may be monitored using an invasive arterial line (A-Line). The A-Line is considered the gold standard in capturing beat-to-beat blood pressure values to detect fluctuations immediately. However, A-Lines are invasive and are associated with known risks including infection, thrombosis, and embolization. Since blood pressure is a dynamic physiologic parameter that changes constantly overtime, and because of the shortcomings of current methods, there is a long-felt need for better methods of continuous blood pressure monitoring.

External radial artery applanation tonometry is a noninvasive, reproducible, and affordable technology that measures blood pressure and the aortic pressure waveform. External radial artery applanation tonometry is performed by applying mild pressure to partially flatten the artery against the relatively rigid bones of the forearm (e.g., the radius). Tonometry means measuring pressure, whereas applanation means to flatten. The radial artery pressure and waveform is then transmitted from the vessel to the sensor and is recorded digitally.

Measurements of the aortic pressure waveform can provide clinically useful information well beyond simple systolic and diastolic readings measured from brachial blood pressure. A trove of information can be gleaned from the shape, amplitude, and duration of the aortic pressure waveform. This information provides insight into the diagnosis and management of many disease states including hypertension, coronary artery disease, sleep apnea, diabetes, and diastolic cardiac dysfunction. The use of external radial artery tonometry is well known, and several studies have shown that arterial pressure waveforms recorded non-invasively by transcutaneous tonometry are largely superimposable over those recorded invasively with an A-Line.

The major or central arteries in the mammalian body are those that are large and primarily found in the chest and abdomen. Examples include the aorta and the major branches of the cardiovascular system, including the brachiocephalic artery, the subclavian arteries, and the left common carotid artery. Peripheral arteries are those arteries found not in the chest or abdomen. Examples include the brachial artery, radial artery, and femoral artery. Central arteries are larger and more elastic in nature, while peripheral arteries are smaller and muscular in structure.

Systolic pressure varies throughout the arterial tree (also called the branching system of arteries) such that an aortic (central) systolic pressure is typically lower than a corresponding brachial pressure, although this difference can vary considerably between individuals. Emerging evidence now suggests that central pressure is better related to future cardiovascular events than is brachial pressure measured by a cuff. Furthermore, anti-hypertensive drugs exert differential effects on both central pressure and peripheral pressure. Thus, basing decisions on central pressure is likely to have important implications for the diagnosis and management of hypertension.

External radial artery tonometry is a well validated and reliable way to record central pressure waves. A peripheral pressure waveform is recorded by tonometry in the radial artery. The peripheral pressure waveform can be used to estimate a corresponding central aortic pressure using a generalized transfer function, identification of the late systolic shoulder of the peripheral pressure waveform, or an algorithm. The FDA has approved derivation and calculation of central pressure indices from a calibrated peripheral pressure wave measured using external radial tonometry.

However, there are limitations to external radial artery applanation tonometry. External radial artery applanation tonometry uses boney tissue to provide support for applanation of the blood vessel with applied pressure. Unfortunately, applanation tonometry tends to be less effective in patients with higher body mass indices because it is difficult to transmit pressure waves through fat. Furthermore, measurements using external radial artery applanation tonometry may be inaccurate if the tonometry device is not placed accurately with respect to the radial artery. Also, tonometric pressure waves measured with external radial artery applanation tonometry should be calibrated against brachial arterial pressure measurements.

### SUMMARY

The inventive technology includes an implantable BP sensing device that tonometrically measures waveforms from a peripheral blood vessel, such as the radial artery, in vivo. The sensing device is configured to be implanted into a patient to measure the patient's BP continually (i.e., regularly) and autonomously over a long period using a novel application based on the principle of applanation tonometry. The sensing device can have different versions, but in all variations, instead of using active, external application of pressure against a rigid boney structure to flatten the artery, the implanted sensing device incorporates a configuration or structure that provides passive, internal applanation either of the sensing device or the blood vessel being measured. The sensing device can be placed to be in direct contact with the targeted blood vessel or can be placed in the soft tissue near the outer wall of the blood vessel (e.g., 1 mm to 10 mm away). Blood pressure is measured by calibrating the generated pressure waveform using brachial arterial pressures.

The implantable BP sensing device addresses problems with external radial artery applanation tonometry and provides a way to record central arterial pressure on a continuous and autonomous basis. Since central arterial pressure is a more accurate predictor of cardiovascular events, the implantable BP sensing device improves the care of patients with hypertension.

In some aspects, the techniques described herein relate to an implantable pressure-sensing device including a substrate configured to secure the implantable pressure-sensing device within 0 mm to 10 mm from a blood vessel, a pressure-sensing element projecting from an outer surface of the substrate and configured to sense blood pressure within the blood vessel, circuitry disposed on the substrate and in electrical communication with the pressure-sensing element and configured to receive data from the pressure-sensing element, and a power management system disposed on the substrate and configured to provide power to the pressure-sensing element and the circuitry.

In some aspects, the techniques described herein relate to an implantable pressure-sensing device further including a coating disposed on the substrate and the pressure-sensing element and hermetically sealing the substrate and the pressure-sensing element, the coating including layers of ceramic and polymer.

In some aspects, the techniques described herein relate to an implantable pressure-sensing device wherein the coating has a thickness of about 1 μm to about 1 mm.

In some aspects, the techniques described herein relate to an implantable pressure-sensing device wherein the coating includes 3 layers to 14 alternating layers of SiOx and Parylene.

In some aspects, the techniques described herein relate to an implantable pressure-sensing device further including an accelerometer disposed on the substrate and configured to detect body movements.

In some aspects, the techniques described herein relate to an implantable pressure-sensing device wherein the pressure-sensing element is a first pressure-sensing element and further including a second pressure-sensing element disposed on the substrate and configured to detect changes in pressure distribution in a mammalian body.

In some aspects, the techniques described herein relate to an implantable pressure-sensing device further including a structure disposed around the pressure-sensing element and projecting from the substrate and configured to transmit pressure waves from the blood vessel towards the pressure-sensing element.

In some aspects, the techniques described herein relate to an implantable pressure-sensing device wherein the structure has a dome shape centered around the pressure-sensing element.

In some aspects, the techniques described herein relate to an implantable pressure-sensing device wherein the structure has a valley shape centered around the pressure-sensing element.

In some aspects, the techniques described herein relate to an implantable pressure-sensing device wherein the structure is shaped in a way to maximize a signal to noise ratio.

In some aspects, the techniques described herein relate to an implantable pressure-sensing device further including an elastomer disposed on the pressure-sensing element and configured to transmit pressure waves to the pressure-sensing element.

In some aspects, the techniques described herein relate to an implantable pressure-sensing device further including a first shape-memory alloy wing mechanically coupled to the substrate and a second shape-memory alloy wing mechanically coupled to the substrate opposite the first shape-memory alloy wing, wherein the first shape-memory alloy wing and the second shape-memory alloy wing are configured to be deployed after the implantable pressure-sensing device is implanted to secure the implantable pressure-sensing device against the blood vessel.

In some aspects, the techniques described herein relate to an implantable pressure-sensing device wherein the substrate is a first substrate and further including a second substrate, wherein the first substrate and the second substrate are part of a rigid housing with an enclosure between the first substrate and the second substrate and wherein the circuitry and the power management system are disposed in the enclosure and the enclosure is filled with an inert polymer filler.

In some aspects, the techniques described herein relate to an implantable pressure-sensing device wherein the inert polymer filler includes epoxy resin.

In some aspects, the techniques described herein relate to an implantable pressure-sensing device wherein the implantable pressure-sensing device has one dimension that is about 10% to about 100% of a width of the blood vessel.

In some aspects, the techniques described herein relate to an implantable pressure-sensing device wherein the implantable pressure-sensing device has a second dimension that is about 25% to about 1000% of the width of the blood vessel.

In some aspects, the techniques described herein relate to an implantable pressure-sensing device wherein the blood vessel is selected from the group consisting of an artery, a vein, a capillary, or a graft.

In some aspects, the techniques described herein relate to an implantable pressure-sensing device wherein the blood vessel is selected from the group consisting of a radial artery, an ulnar artery, a brachial artery, or a sub-clavian artery.

In some aspects, the techniques described herein relate to an implantable pressure-sensing device wherein the blood vessel is a blood vessel in a lower limb.

In some aspects, the techniques described herein relate to an implantable pressure-sensing device wherein the blood vessel is a great vessel.

In some aspects, the techniques described herein relate to an implantable pressure-sensing device including: a substrate configured to be secured within 0 mm to 10 mm of a blood vessel and a pressure-sensing element projecting from the substrate and configured to make measurements of blood pressure in the blood vessel when the substrate is secured within 0 mm to 10 mm of the blood vessel.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1A illustrates a cross-section of a first implantable sensing device.

FIG. 1B illustrates a plan view of the sensing device in FIG. 1A.

FIG. 1C illustrates the sensing device in FIG. 1A implanted in contact with or in proximity to (e.g., within 0 mm to 10 mm of) a blood vessel.

FIG. 2A illustrates a cross-section of an implantable sensing device with a frustoconical structure to focus the pulse wave onto the pressure-sensing device's pressure-sensing element.

FIG. 2B illustrates a plan view of the sensing device in FIG. 2A.

FIG. 2C illustrates the sensing device in FIG. 2A implanted in contact with or in proximity to (e.g., within 0 mm to 10 mm of) a blood vessel.

FIG. 3A illustrates a cross-section of an implantable sensing device with a valley-shaped structure to focus the pulse wave onto the device's pressure-sensing element.

FIG. 3B illustrates a plan view of the sensing device in FIG. 3A.

FIG. 3C illustrates the sensing device in FIG. 3A implanted in contact with or in proximity to (e.g., within 0 mm to 10 mm of) a blood vessel.

FIG. 4A illustrates a cross-section of the sensing device with a polymer protrusion or dome disposed on the pressure-sensing element.

FIG. 4B illustrates a plan view of the sensing device in FIG. 4A.

FIG. 4C illustrates the sensing device in FIG. 4A implanted in contact or in proximity to an blood vessel.

FIG. 5A illustrates a cross-section of the sensing device with a balloon filled with liquid disposed around the pressure-sensing element.

FIG. 5B illustrates a plan view of the sensing device in FIG. 5A.

FIG. 5C illustrates the sensing device in FIG. 5A implanted in contact with or in proximity to (e.g., within 0 mm to 10 mm of) a blood vessel.

FIG. 6 illustrates a cross-section of the sensing device with a balloon filled with liquid coupled to the sensing element implanted posterior to an blood vessel with the balloon on top of bone (e.g., the radius bone).

FIG. 7A illustrates a cross-section of the sensing device with shape-memory alloy wings.

FIG. 7B illustrates a plan view of the sensing device in FIG. 7A.

FIG. 7C illustrates deployment of the sensing device in FIG. 7A in contact with or in proximity to (e.g., within 0 mm to 10 mm of) a blood vessel.

FIG. 8A illustrates a modified Kelvin-Voigt model of the viscoelastic behavior of a blood vessel.

FIG. 8B illustrates hysteresis in stress-strain measurements of a blood vessel wall illustrating the blood vessel wall's viscoelastic behavior according to the modified Kelvin-Voigt model in FIG. 8A.

FIG. 9 shows a pulse pressure waveform analysis.

FIG. 10 illustrates a cross-section of a sensing device used externally.

FIG. 11A is a plan view photo of a wired sensing device used externally.

FIG. 11B is a plan view photo of a wired sensing device used externally.

FIG. 11C is a photo of the sensing device in FIGS. 11A and 11B being used externally.

FIG. 12 is a cross-section of a sensing device with two sensors.

### DETAILED DESCRIPTION

Conventional external applanation tonometry sensors, where the sensor is placed against a patient's skin, can be inaccurate and unreliable. Since fat underneath the skin does not readily transmit pressure waves, measurements by conventional applanation tonometry sensors can be inaccurate. These flaws may be exacerbated in patients with higher body mass indices. Patients with higher body mass indices are often also at higher risk for cardiovascular disease and hypertension and would benefit more from accurate and reliable blood pressure (BP) measurements.

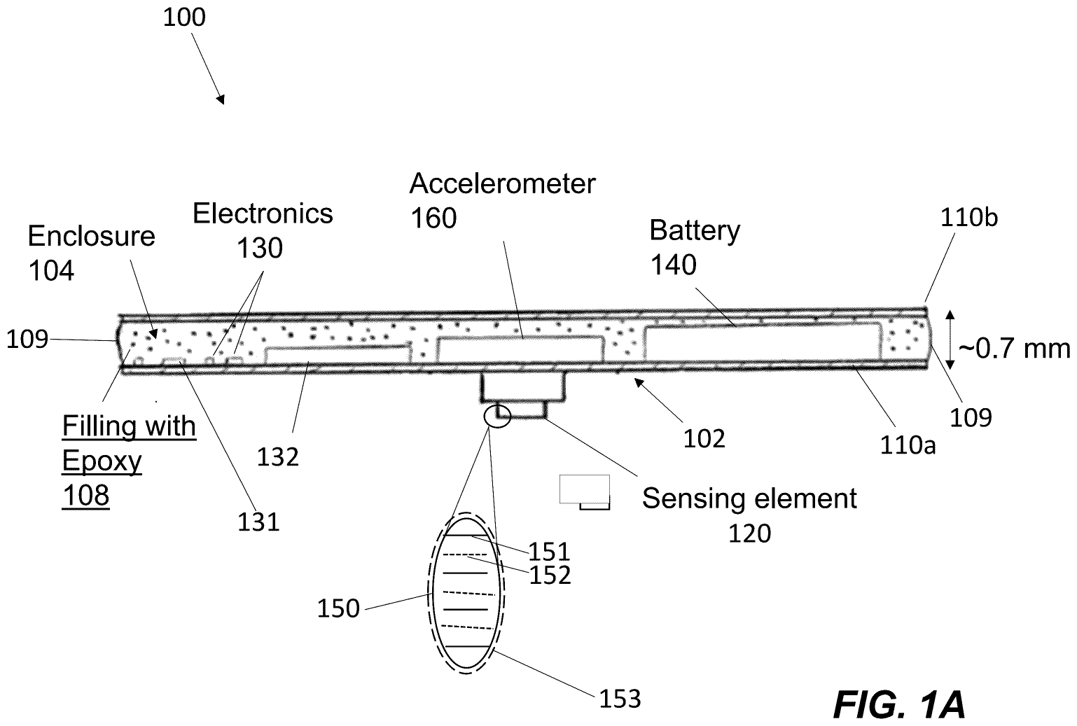

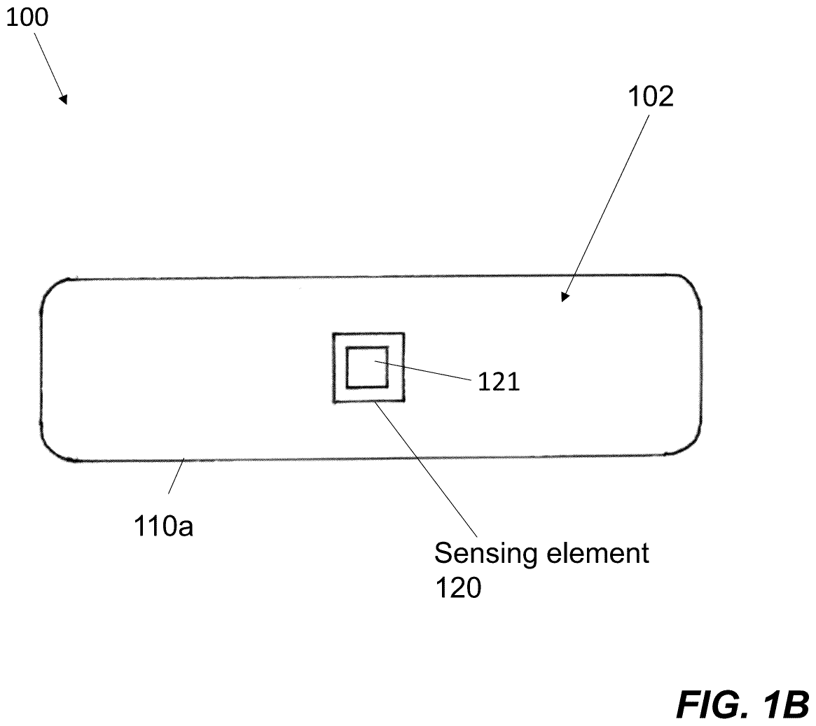

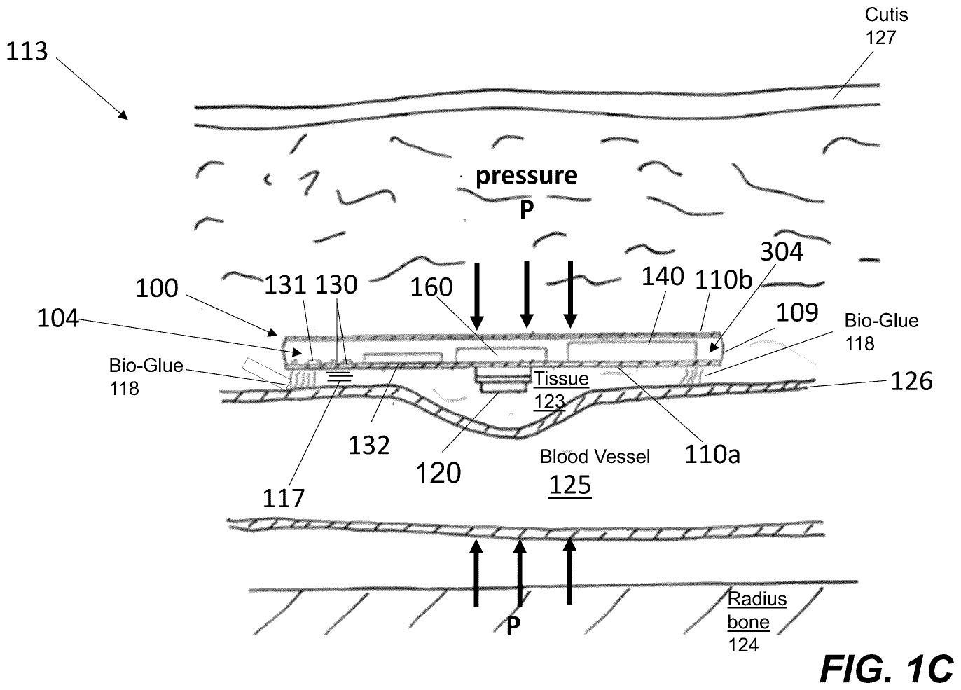

FIGS. 1A-7C show various versions and views of implantable BP sensing devices (also called a sensing device or a pressure-sensing device) that has a pressure-sensing element configured to measure cardiovascular pressure using applanation tonometry principles. The sensing device is configured to be implanted into a mammalian body so that it is disposed on or adjacent (e.g., 1 mm to 10 mm away from) a blood vessel. For example, the sensing device may be placed in the soft tissue near the outer wall of the blood vessel at a distance of 0 mm, 1 mm, 2 mm, 3 mm, 4 mm, 5 mm, 6 mm, 7 mm, 8 mm, 9 mm, 10 mm, or any value in between said values, from the blood vessel. The blood vessel may be any type of arterial structure where measurement of pressure is relevant to disease management. For example, the arterial structure may include a natural blood vessel (e.g., an artery, vein, or capillary) or a synthetic/artificial blood vessel (e.g., a graft). The artery may include a radial artery, an ulnar artery, a brachial artery, a sub-clavian artery, a renal artery, or the abdominal aorta, for example. The blood vessel may also include a great vessel (e.g., the inferior vena cava, the superior vena cava, a pulmonary artery, a pulmonary vein, or the aorta). The arterial structure may be a natural or synthetic blood vessel in an upper extremity (e.g., an arm), a lower extremity (e.g., a leg), the trunk, and/or the head and neck. The sensing device may assist in disease management of hypertension, limb ischemia, or diseases of the aorta, including complications of aortic replacement (e.g., an aortic endograft leak). The pressure-sensing device is configured to measure cardiovascular pressure on a continuous and autonomous basis after it is implanted in a patient.

Once implanted, the sensing device continually and automatically measures BP using applanation tonometry principle. As the heart beats, it pushes blood through the blood vessel, causing the blood vessel to expand against the pressure-sensing element. The sensing device itself may be rigid or include one or more rigid components such that the blood vessel flattens against it as it expands. The amount of force that flattens the blood vessel against the sensing device is directly proportional to the blood pressure in the blood vessel. This proportional relationship can be expressed as: blood pressure ˜(contact force)/(area of contact). The proportionality constant that relates this proportional relationship depends on the thickness and biomechanical properties of the wall of the blood vessel, which is different for every individual. These properties are discussed in more detail below. The proportionality constant can be determined by calibrating the implanted pressure-sensing device. This calibration can be repeated as desired.

Alternatively, the sensing device 100 may be soft or flexible. The softness and/or flexibility of the sensing device 100 may be based on the softness and/or flexibility of the blood vessel and/or surrounding tissue. Preferably the sensing device 100 is more rigid than the blood vessel and/or surrounding tissue.

The pressure-sensing device is configured to be able to be placed into the body on top of a central and/or peripheral blood vessel percutaneously or in an open surgically exposed site. For example, for the peripheral radial artery, the implant can be placed percutaneously or by direct exposure of the vessels. An advantage of implanting the sensing device percutaneously on or adjacent to the radial artery is that the radial artery is more accessible for implantation than some other sites in the mammalian body and the implantation procedure can be a simple outpatient procedure. For major or central arteries, the implanted pressure-sensing device can be delivered or implanted either percutaneously or surgically in an open manner onto a major natural or synthetic blood vessel (vascular graft).

FIG. 1A illustrates a cross-section of an inventive version of a pressure-sensing device 100. FIG. 1B illustrates a plan view of the sensing device 100 in FIG. 1A. The sensing device 100 includes a pressure-sensing element 120 that is disposed on the sensing device's outer surface 102 and that measures changes in pressure using applanation tonometry principle. In some versions, the sensing device 100 includes two substrates 110a and 110b sandwiched together to form an enclosure or housing 104. The housing 104 may have a thickness between the two substrates 110a and 110b of about 0.5 mm to about 1.5 mm (e.g., about 0.7 mm). The housing 104 may have a width of about 10% to about 100% of a width of the blood vessel 125 at the target implantation site (e.g., a width of 0.2 mm, 0.5 mm, 0.75 mm, 1 mm, 2 mm, 5 mm, 10 mm, 25 mm, 50 mm, or any value between 0.2 mm and 50 mm). The housing 104 may also have a length of about 25% to about 1000% of the width of the blood vessel 125 (e.g., a length of 0.5 mm, 1 mm, 2 mm, 5 mm, 10 mm, 25 mm, 50 mm, or any value between 0.5 mm and 50 mm). The blood vessel 125 may have a diameter from about 1 mm to about 6 cm. Having one dimension of the housing 104 longer than another facilitates introduction and placement of the sensing device 100 on or near the blood vessel 125, where the longer dimension of the sensing device 100 can be aligned with the length of the blood vessel 125. In other versions, the sensing device 100 includes one flat or curved substrate 110a/110b and a coating 150 disposed over the substrate 110a/110b to form a hermetical seal. The coating 150 may be disposed over the entire sensing device 100. And in still other versions, the sensing device includes a flat or curved substrate 110a/110b and a casing or housing disposed around the substrate 110a/110b.

One of the substrates 110a of the sensing device 100 may be a printed circuit board (PCB) (or another suitable electronics carrier) upon which circuitry including electronic components 130 are mounted and electrically connected with conductive links. The printed circuit board may be made of a rigid polymer or a ceramic. Another substrate 110b may be a cover. As an example, the cover may be made of silicon, a material commonly used in microtechnology production, to reduce production costs. As another example, the cover may be another non-conductive material, such as a ceramic, polymer, or metal. The housing 104 may be at least partially filled with an inert polymer filler 108 (e.g., epoxy resin) to add mechanical rigidity to the pressure-sensing device 100 and to help seal the housing 104 and prevent liquid infiltration into the housing 104. The inert polymer filler 108 is a biocompatible material that can be applied in liquid form, and, in some cases, cures to become a solid material. If the sensing device 100 includes an epoxy resin filler 108, then the housing 104 may not need side walls 109. In some embodiments, the housing 104 may be filled with a softer epoxy resin filler to allow for a softer and/or more flexible sensing device 100. Alternatively, the housing 104 may be filled with a silicone gel. The substrates 110a and 110b can be made of metal or another material, such as a dielectric material. However, using nonmetallic substrates reduces or eliminates interference by the substrates 110a and 110b with any wireless communication and wireless power charging functionalities in the pressure-sensing device 100.

The rigidity of the pressure-sensing device 100 is influenced by the shape of the sensing device 100, the sensing device's 100 length-to-width ratio, the thickness of the sensing device 100, and the mechanical properties of the device's substrate(s) 110a and 110b and filling material 108. The rigidity of the pressure-sensing device 100 may help secure the pressure-sensing element 120 against or at a fixed distance away from a blood vessel 125 in the mammalian body, as explained in more detail below, and may be useful for tonometry. The rigidity of the pressure-sensing device 100 may also protect the electronic components 130 in the sensing device from overbending and breakage and from outside impacts. The sensing device 100 also has a certain ductility so that it does not easily shatter from an outside impact. The ductility may be imparted by the filling material (e.g., an epoxy resin).

The pressure-sensing device 100 may also be soft and/or flexible such that the sensing device 100 may bend. The sensing device 100 may bend during a measurement. Alternatively, the sensing device 100 may bend as a result of movement of the mammalian body in which the sensing device 100 is placed. A soft and/or flexible pressure-sensing device 100 may allow for placement of the pressure-sensing device 100 near a structure in the body where there is motion such as a joint (e.g., a wrist joint, a leg joint, and/or an arm joint). For example, the pressure-sensing device 100 may be placed on the radial artery near the wrist joint. Any signal noise generated by the movement of the mammalian body may be accounted for using the accelerometer and processing of the data.

The pressure-sensing element 120 is mounted to and protrudes or projects from the outer surface of the substrate 110a (e.g., a printed circuit board). The pressure-sensing element 120 may include a microelectromechanical (MEMS) sensing element, capacitive sensing element, piezoelectric sensing element, or another suitable pressure-sensing element. The pressure-sensing element 120 measures pressure in the range of about 40 mm Hg to about 250 mm Hg. The pressure-sensing element 120 is electrically connected to electronic components 130 in the housing via conductive links running through the substrate 110a (e.g., a printed circuit board).

The pressure-sensing element 120 has a sensing surface 121 that measures perpendicularly applied force over a known area. When the sensing device 100 is deployed in the body so that the pressure-sensing element 120 (which may be coated with the coating 150 described in more detail below) is in direct contact with the outer wall 126 of a blood vessel 125 or in proximity to the outer wall 126 of the blood vessel 125, the pressure-sensing element 120 measures the pressure or force of blood pushing against the blood vessel walls. Pumping by the heart results in the development of pressure in the blood vessels 105 and this is the pressure which is measured by the pressure-sensing element 120. The pressure-sensing element 120 is configured to measure waveform pressure through the cardiac cycle. The pressure waveform that is observed by the sensing device 100 reflects the events of the cardiac cycle, and includes the peak systolic pressure, aortic valve closure (dicrotic notch), and the diastolic pressure.

FIG. 1C illustrates the pressure-sensing device in FIG. 1A implanted against a blood vessel 125. The sensing device 100 is implanted in a mammalian body 113 beneath the cutis 127 so that the pressure-sensing element 120 is disposed in direct contact with the outer wall 126 of the blood vessel 125 or at a fixed distance away from the blood vessel 125. Preferably, the pressure-sensing element 120 is aligned to the center of the blood vessel 125 so that the pressure-sensing element 120 is exposed to a greater portion of the blood pressure wave. In some versions, the implantation site may be a portion of the blood vessel 125 disposed adjacent to bone 124. Positioning the pressure-sensing device 100 on or in proximity to a portion of the blood vessel 125 adjacent to bone 124 may improve pressure measurements because the bone 124 helps keep the pressure-sensing device 100 in a fixed position. For example, the blood vessel 125 may be the radial artery and the bone 124 may be the radius bone.

When the sensing device 100 is implanted at a fixed distance from the blood vessel 125, the sensing device 100 is implanted in proximity to the blood vessel 125 with space and/or soft tissue 123 between the pressure-sensing device 100 and the outer wall of the blood vessel 126. Preferably the sensing device 100 remains in its implanted position at a fixed distance from the blood vessel 125. In some embodiments, the pressure sensing device 100 may move slightly (e.g., by millimeters or less) after implantation due to movement of the person, fibrotic tissue growth, etc. Some of the soft tissue 123 between the sensing device 100 and the outer wall 126 of the blood vessel 125 may be fibrotic tissue that forms after the sensing device 100 is implanted. The distance between the pressure-sensing element 120 and the outer wall 126 of the blood vessel 125 may be about 10 mm or less. When the sensing device 100 is implanted within a certain distance from the blood vessel 125 (e.g., within 10 mm or less), the sensing device 100 may also be implanted with one or more layers of silicone 117 between the pressure-sensing device 100 and the outer wall of the blood vessel 126. The one or more layers of silicone 117 may help secure the sensing device 100 at its implant location (i.e., at a fixed distance from the blood vessel 125).

During implantation, a bio-glue 118 can be used to adhere portions of the pressure-sensing device's outer surface 102 to portions of the outer wall 126 of the blood vessel 125 or surrounding tissue 123, with the projecting pressure-sensing element 120 pointed toward or in direct contact with the outer wall 126 of the blood vessel 125. The bio-glue 118 may include a collagen material. The bio-glue 118 helps keep the sensing device 100 positioned with respect to the blood vessel 125 so that the pressure-sensing element 120 applies a steady force against the wall 126 of the blood vessel 125 to applanate a small portion of the wall 126 of the blood vessel 125 in order to measure the blood pressure. Over time (e.g., in about 4 to 8 weeks), fibrotic tissue may build up around the sensing device 100 and help keep the sensing device 100 fixed in position on the blood vessel 125 over the long-term. In some versions, the bio-glue 118 dissolves in the body 113 over a period of 4 to 8 weeks, and the fibrotic tissue helps keep the sensing device 100 fixed in its implanted position after the bio-glue 118 dissolves. Preferably the sensing device 100 remains in its implanted position after the bio-glue 118 dissolves. Fibrosis does not affect the ability of the sensing device 100 to accurately measure cardiovascular pressure. Alternatively, the sensing device 100 can be anchored with suture techniques to maintain position relative to the targeted blood vessel.

The electronic components 130 disposed in the housing and mounted to the inner surface of the substrate 110a (e.g., a printed circuit board) may include an accelerometer 160 and a power source 140. The inert polymer filler 108 may at least partially surround the electronic components 130 in the housing 104. The accelerometer 160 detects or measures body movements (e.g., movements of the arm in which the pressure-sensing device 100 is implanted) and the accelerometer data are used in processing the pressure data to reduce artifacts associated with body movements. The accelerometer data are used to differentiate between body movements and blood vessel pressure waves. Accelerometer data is used to detect body movement and related blood vessel pressure artifacts. The accelerometer data may subsequently be used for compensating these artifacts. The power source 140 may be a primary battery or a rechargeable battery. Preferably, the power source 140 is a rechargeable battery that is configured to be charged wirelessly so that the implanted sensing device 100 can operate for extended periods while implanted. In some versions, the sensing device 100 may include one or more antennas 131 for data communication and wireless charging. The antenna 131 may be a strip or coil of conductive metal (e.g., gold or copper) disposed on the substrate 110a (e.g., a printed circuit board). The antenna(s) 131 may be driven or arranged to enable almost omnidirectional characteristics for wireless charging and communication.

The pressure-sensing device includes a controller 132. The controller 132 may be an application-specific integrated circuit (ASIC). The ASIC 132 receives signals from the pressure-sensing element(s) 120 and the accelerometer 160. The ASIC 132 receives and processes signals from the pressure-sensing element 120. For instance, the ASIC 132 (or a separate analog-to-digital converter) may convert the signals from the analog domain to the digital domain and time average or filter the digital data to reduce noise. The ASIC 132 may also reduce or substantially remove artifacts in the data related to body movement using data from the accelerometer 160, which tracks body movement. The ASIC 132 may include one or more forms of memory. For example, the ASIC 132 may include volatile memory (e.g., RAM) that is used for controlling electrical components and processing data. The ASIC 132 may have a flash memory that stores data for some processing. For example, the flash memory may be used to perform signal averaging of pressure-sensing data received from the pressure-sensing element 120.

The ASIC 132 may also include a power controller to manage electrical power usage by the pressure-sensing device 100. For example, the ASIC 132 may determine the charge state of a rechargeable battery 140 in the sensing device 100 that provides electrical power to the electrical components 130 in the sensing device 100 and indicate to the patient or healthcare provider when the battery 140 needs to be recharged (e.g., by sending a wireless notification to an external device). The ASIC 132 may also manage any wireless communication components in the sensing device 100. For example, the ASIC 132 may adjust electrical characteristics of an antenna 131 circuit in the sensing device 100 in order to increase or maximize wireless coupling efficiency with an external device.

The pressure-sensing device 100 may be coated with a coating 150 that forms a hermetic seal. The coating 150 is disposed on the outer surfaces of the pressure-sensing device, including the pressure-sensing element 120 itself. The coating 150 is made of a deformable material that allows pressure to be communicated from the environment to the pressure-sensing element 120. The coating 150 is thin and flexible enough that it transmits pressure exerted in the environment outside of the pressure-sensing device 100 to the sensing element 120. In some versions, the coating 150 is a multilayer coating formed of alternating layers of ceramic (e.g., silicon dioxide 151) and polymer (e.g., Parylene-C 152 (a chlorinated poly(para-xylylene) polymer)). Instead of, or in addition to, Parylene-C, Parylene-F (a fluorinated poly(para-xylylene) polymer) may be used to form the coating 150. Optionally, a thin and deformable silicone gel 153 (e.g., silastic) coating may be disposed on the sensing device 100 on an inner or outer surface of the multilayer coating 150. For example, the silicone gel coating 153 may be disposed between the filling material 108 and the multilayer coating 150 to act as an adhesion layer that helps the multilayer coating 150 to stick fast to the filling material 108.

In some embodiments the total thickness of the coating 150 is in the range of 1 μm to 1 mm (e.g., 1 μm, 5 μm, 10 μm, 14 μm, 20 μm, 25 μm, 50 μm, 100 μm, 150 μm, 200 μm, 500 μm or 1 mm). As an example, each layer may be about 500 nm to about 2 μm thick, the multilayer coating 150 may alternate between the SiO₂ layer 151 and Parylene layer 152 with 3 layers to 7 layers of each (6 layers to 14 layers total), and the multilayer coating 150 may have a total thickness of about 6 μm to about 14 μm.

Some versions of implantable pressure-sensing device 100 may have a structure and/or channel disposed around or on the pressure-sensing element 120 and projecting from an outer surface of the sensing device 100. The structure and/or channel increases the contact surface area between the pressure-sensing device and the blood vessel and/or directs/transmits pressure waves from the blood vessel towards the pressure-sensing element 120.

The structure and/or channel may have any of several shapes and sizes and may be made of any of several materials selected to direct/transmit pressure waves towards the pressure-sensing element 120 and/or increase the contact surface area between the pressure-sensing device and the blood vessel. The structure and/or channel directs/transmits pressure waves toward the pressure-sensing element 120 like a funnel. The structure and/or channel's shape may be valley-shaped (e.g., U-shaped, V-shaped, or a combination thereof), cube-like, cuboid, ellipsoidal, toroidal, hemispherical, cylindrical, cone-like, tetrahedral, or any combination thereof, with rounded edges. The structure and/or channel may have a concavity, depression, hollow and/or channel with a shape that is valley-shaped (e.g., U-shaped, V-shaped, or a combination thereof), conical, saddle-shaped, pyramidal, ellipsoidal, hyperboloidal, paraboloidal (e.g., elliptic paraboloidal, parabolic cylindrical, or hyperbolic paraboloidal), or a combination thereof. Preferably the structure is shaped in a way to increase or maximize the signal-to-noise ratio (SNR) of the pressure pulse waveform generated by the pressure-sensing element 120 in response to the BP. For example, the structure may be in the shape of a dome, a circle, and/or an oval. Alternatively, the structure may be in the shape of a cone, a frustoconical shaped structure, and/or a valley shaped structure. If the concavity, depression, or hollow is present in the structure and/or channel, the concavity, depression, or hollow is centered around or over the pressure-sensing element 120.

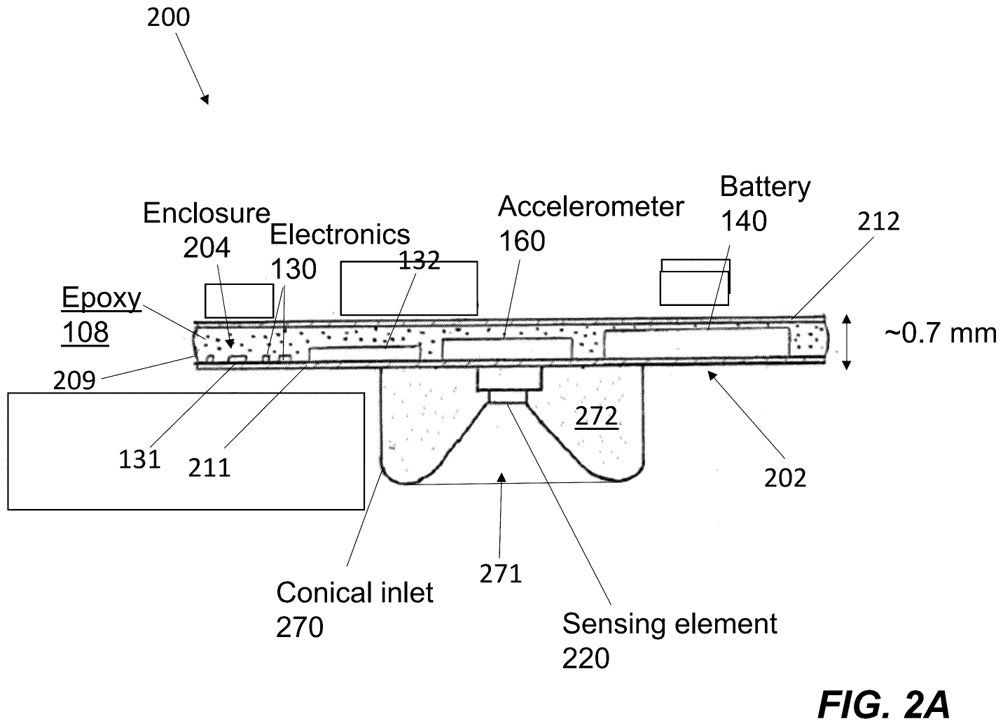

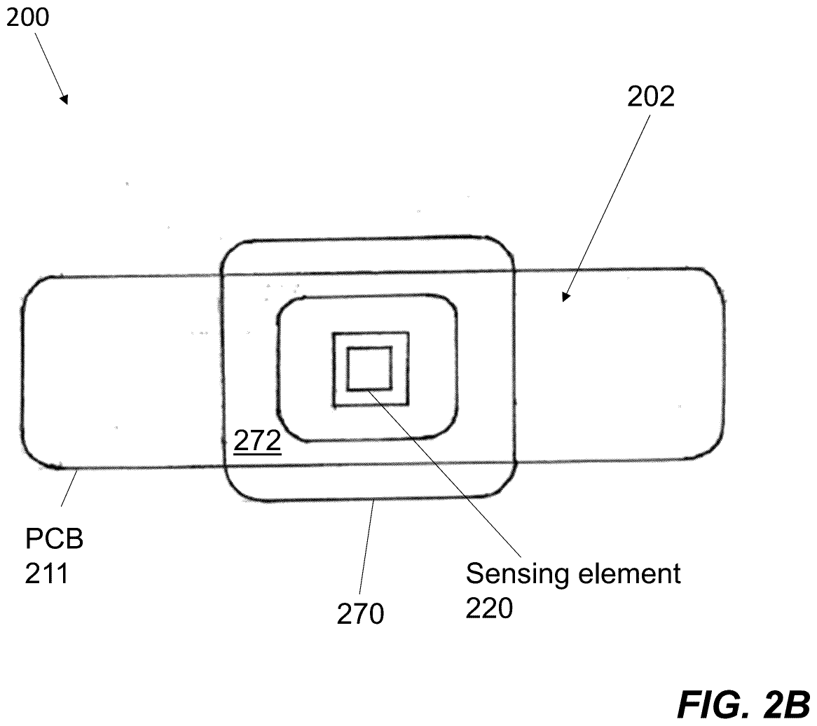

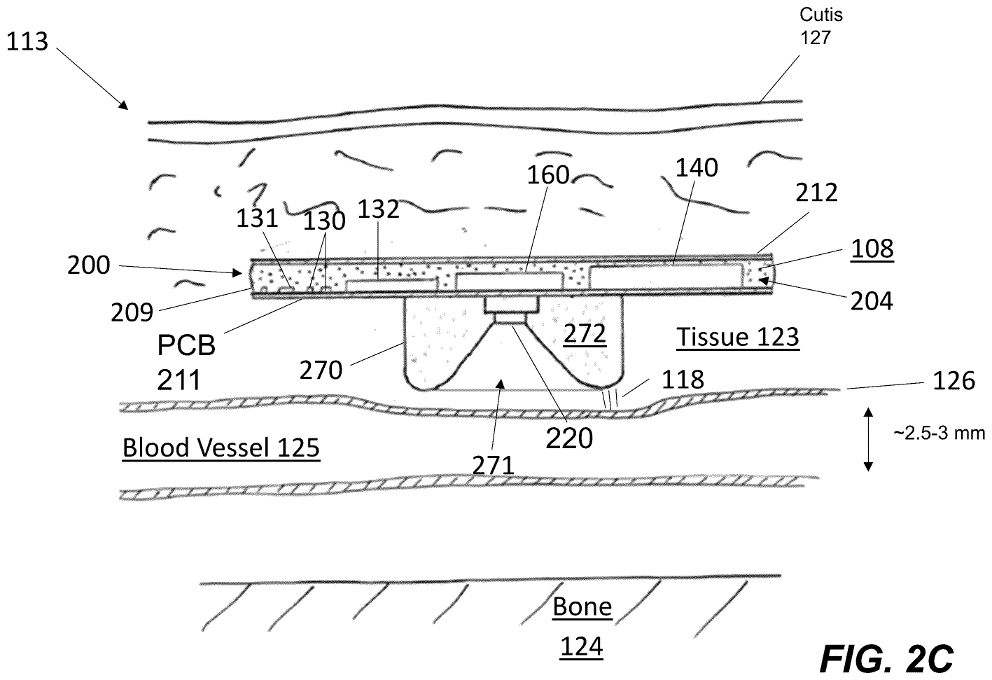

The structure and/or channel has lateral dimensions that may range from matching the lateral dimensions of the pressure-sensing element 120 to being about 10% to about 200% of the dimensions of the substrate 110a or 110b on which the structure and/or cavity is disposed, or any value in between (e.g., 12%, 50%, 100%, 150%, or 200%). The structure and/or channel may have lateral dimensions of about 0.5 mm to about 4 mm (e.g., 0.5 mm, 1 mm, 2 mm, or 4 mm). In some versions, the structure and/or cavity is made of a single material or substantially homogeneous matrix of materials. The material(s) of the structure and/or channel are flexible and substantially incompressible. As an example, the material(s) of the structure and/or channel may have mechanical properties similar to or the same as silastic (i.e., a tensile strength of about 3 MPa to about 6 MPa, a hardness of about 30 shore A to about 60 shore A, and an elongation at break of about 300% to about 600%). In other versions, the structure and/or cavity has an outer solid coating 150 or shell and is filled with a liquid, gel, or another fluid-like substance. The outer solid coating 150 forms a hermetic seal and may be a multilayer composite of alternating layers of Parylene 152 and SiOx 151. For example, the outer solid layer may include 3 layers each (6 layers total) of Parylene 152 and SiOx 151, with the Parylene layers 152 each having a thickness of about 1 μm to about 2 μm and the SiOx layers 151 each having a thickness of about 500 nm. The liquid or gel filler inside of the outer solid coating 150 may be an incompressible silicone material with a high water content. The material(s) forming the structure can be inert biocompatible polymers. Versions of structures and/or cavities are described in more detail below and with regard to FIGS. 2A to 5C.

FIG. 2A illustrates a cross-section of an implantable pressure-sensing device 200 with a conical structure 270 centered around the pressure-sensing element 220 to focus the pulse wave from the blood vessel 125 to the pressure-sensing element 220. FIG. 2B illustrates a plan view of the sensing device in FIG. 2A. One of the substrates of the sensing device 200 is a printed circuit board (PCB) 211 and the other substrate of the sensing device 200 is a cover 212. The PCB 211 and cover 212 may be sandwiched together to form an enclosure or housing 204, as described above. The housing 204 may be at least partially filled with an inert polymer filler 108 (e.g., epoxy resin) to add mechanical rigidity to the pressure-sensing device 200 and to help seal the housing 204 and prevent liquid infiltration into the housing 204. If the sensing device 200 includes an epoxy resin filler 108, then the housing 204 may not need side walls 209. The conical structure 270 projects from the outer surface 202 of the printed circuit board 211. The structure 270 is made of an inert biocompatible polymer 272. The structure's 270 concavity 271 is centered around and surrounds the pressure-sensing element 220 to focus the pulse wave onto the pressure-sensing element 220. FIG. 2C illustrates the sensing device 200 in FIG. 2A implanted against an blood vessel 125 and preferably aligned so that the conical structure 270 is centered on the blood vessel 125. The structure 270 creates a larger contact area with the blood vessel 125 to channel the pressure wave toward the pressure-sensing element 220. Like the pressure-sensing device 100 in FIG. 1C, the pressure-sensing device 200 in FIG. 2C may be attached to the blood vessel 125 using bio-glue 118 disposed on the sensing device's substrate (i.e., PCB 211) and/or structure 270 or using sutures. Additionally, like the pressure-sensing device 100 in FIGS. 1A-1C, the pressure-sensing device 200 may be coated with the coating 150 (including the alternating layers of Parylene 152 and SiOx 151) and the silicone gel 153.

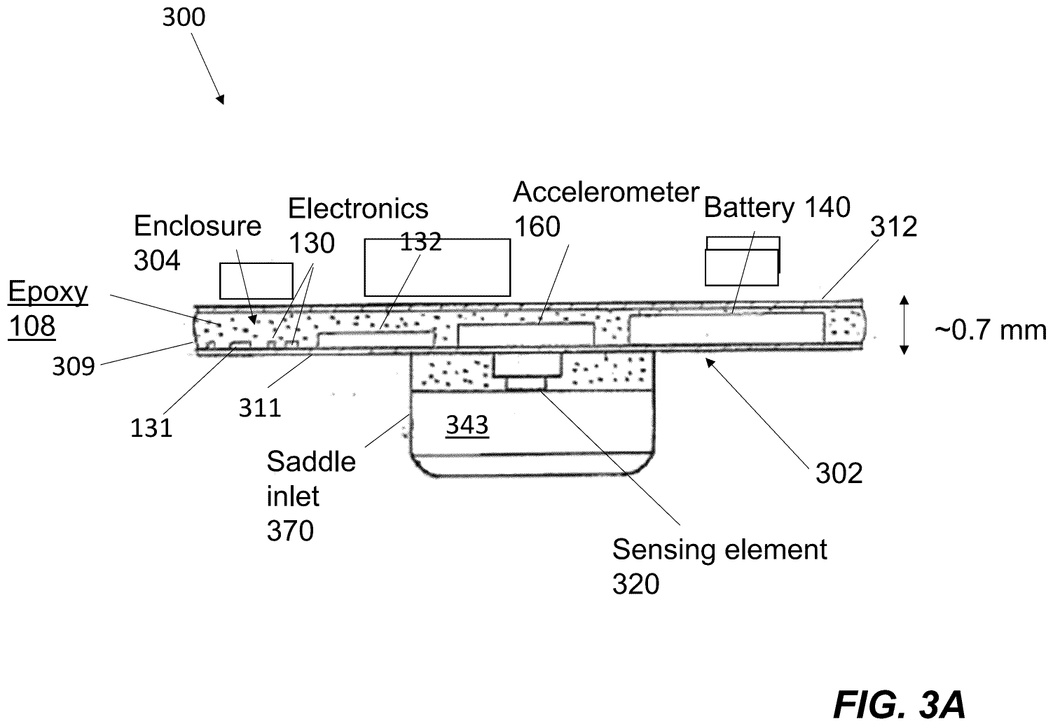

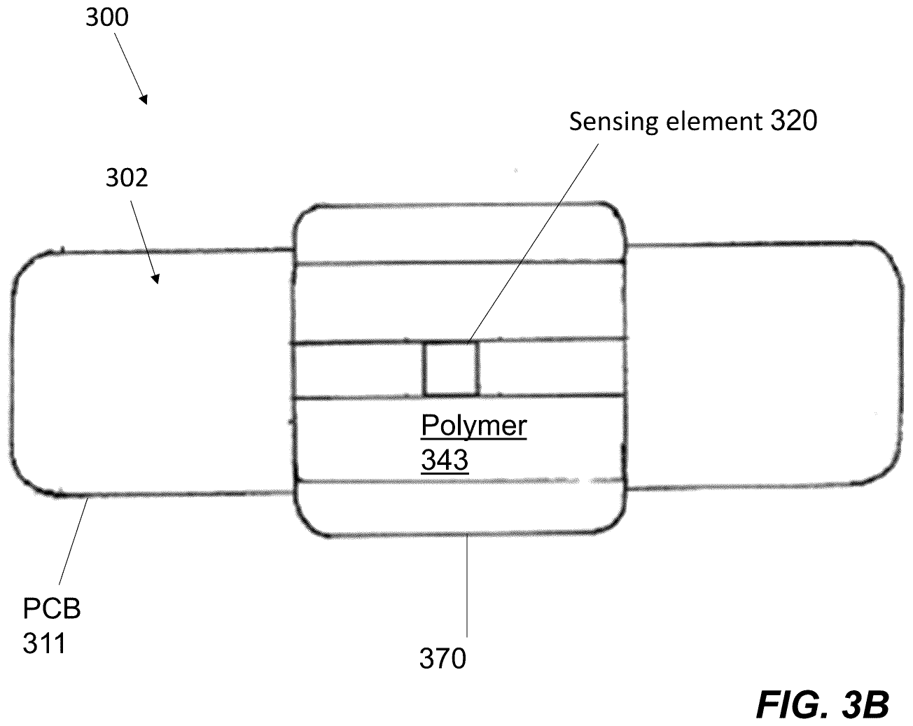

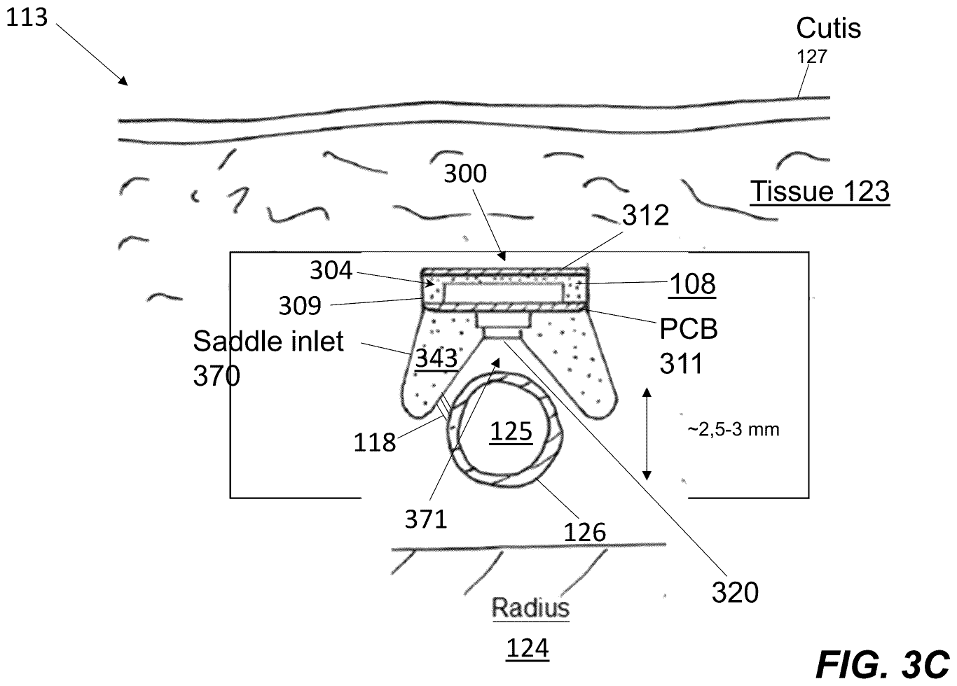

FIG. 3A illustrates a cross-section of an implantable pressure-sensing device 300 with a valley-shaped structure 370 to focus the pulse wave onto the sensing device's pressure-sensing element 320. FIG. 3B illustrates a plan view of the sensing device 300 in FIG. 3A. One of the substrates of the sensing device 300 is a printed circuit board (PCB) 311 and the other substrate of the sensing device 300 is a cover 312. The PCB 311 and cover 312 may be sandwiched together to form an enclosure or housing 304, as described above. The housing 304 may be at least partially filled with an inert polymer filler 108 (e.g., epoxy resin) to add mechanical rigidity to the pressure-sensing device 300 and to help seal the housing 304 and prevent liquid infiltration into the housing 304. If the sensing device 300 includes an epoxy resin filler 108, then the housing 304 may not need side walls 309. The structure 370 projects from the outer surface 302 of the printed circuit board 311. The structure 370 is made of an inert biocompatible polymer 343. The valley-shaped structure 370 is centered around and surrounds the pressure-sensing element 320 to focus the pulse wave onto the pressure-sensing element 320. FIG. 3C illustrates the sensing device 300 in FIG. 3A implanted against a blood vessel 125. The valley shape 371 is parallel to the blood vessel 125 and straddles the blood vessel 125 to focus the pulse wave onto the sensing device's pressure-sensing element 320. Like the sensing device 100 in FIG. 1C, the sensing device 300 in FIG. 1C may be attached to the blood vessel 125 using bio-glue 118 or other fixation such as suturing to adjacent tissue 123. Additionally, like the pressure-sensing device 100, the pressure-sensing device 300 may be coated with the coating 150 (including the alternating layers of Parylene 152 and SiOx 151) and the silicone gel 153.

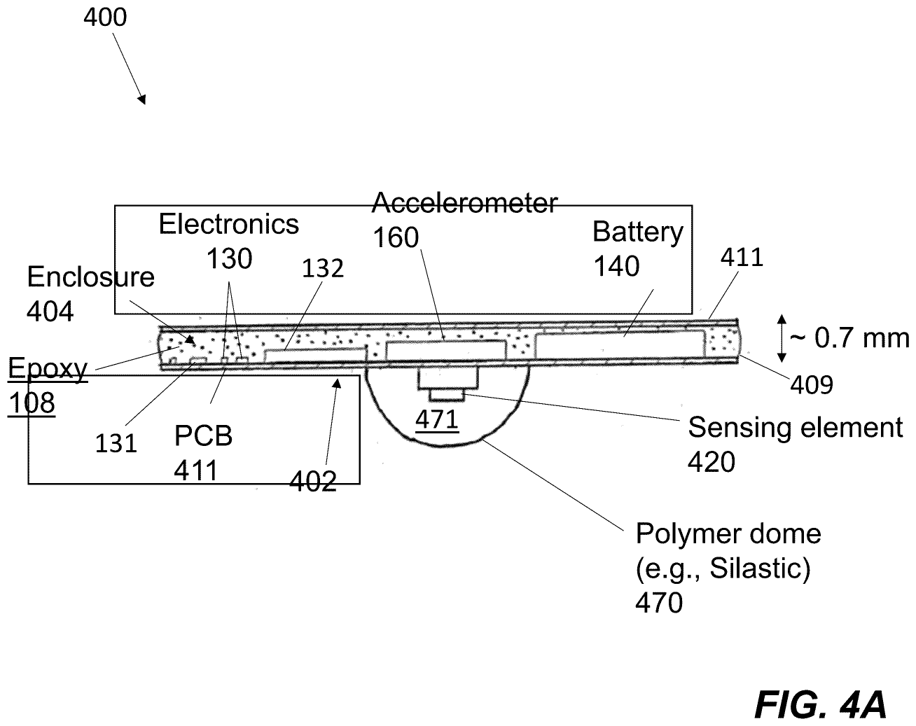

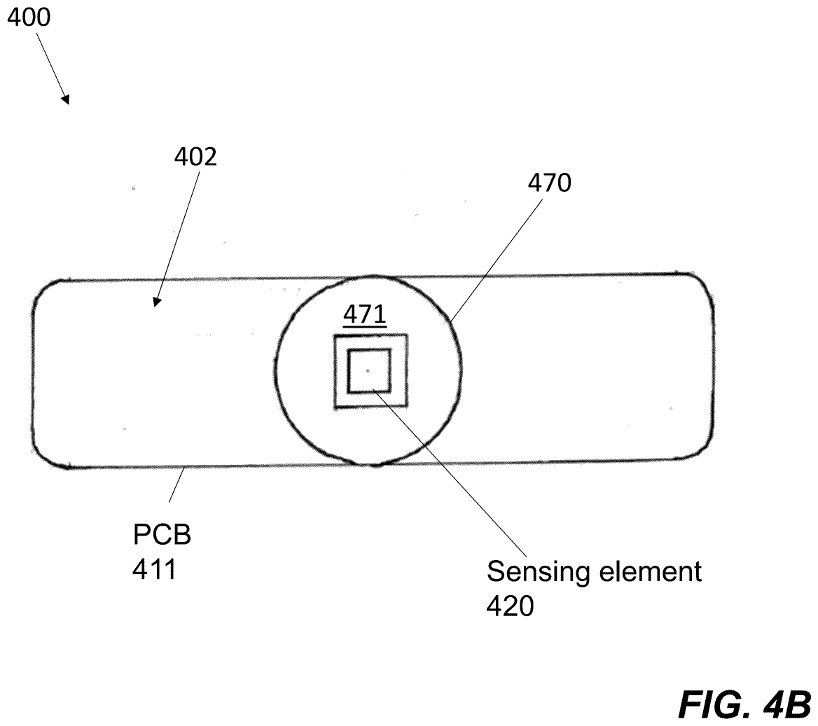

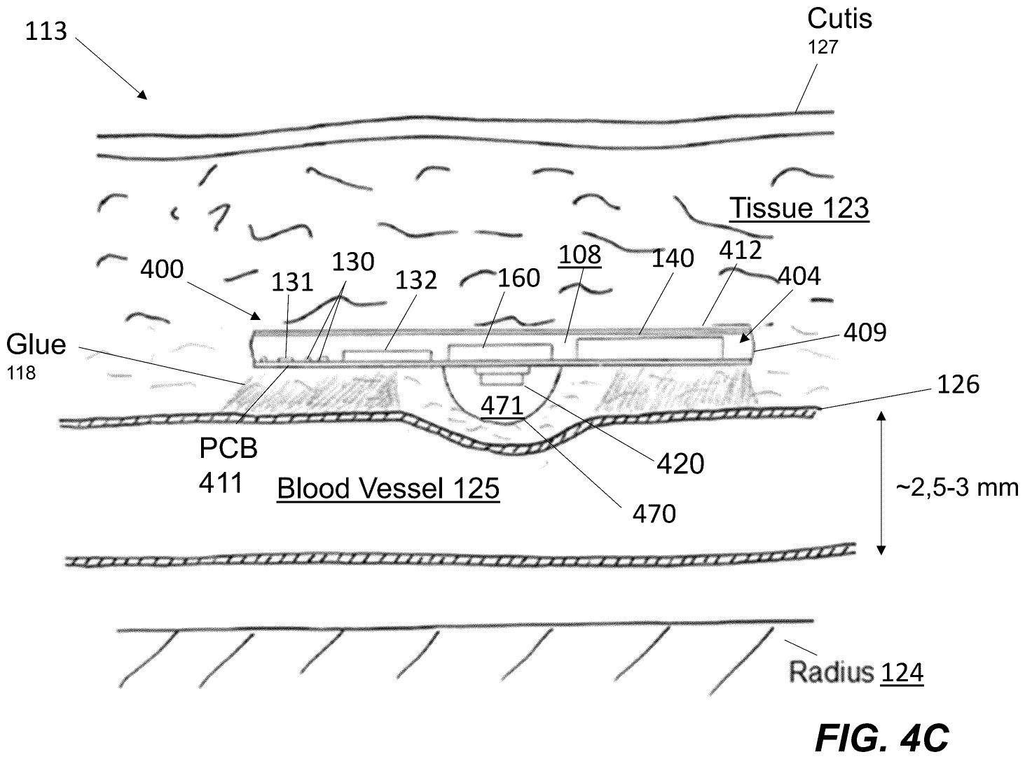

FIG. 4A illustrates a cross-section of an implantable pressure-sensing device 400 with a polymer elastomer protrusion structure 470 disposed on the pressure-sensing element 420. FIG. 4B illustrates a plan view of the sensing device 400 in FIG. 4A. One of the substrates of the sensing device 400 is a printed circuit board (PCB) 411 and the other substrate of the sensing device 400 is a cover 412. The PCB 411 and cover 412 may be sandwiched together to form an enclosure or housing 404, as described above. The housing 404 may be at least partially filled with an inert polymer filler 108 (e.g., epoxy resin) to add mechanical rigidity to the pressure-sensing device 400 and to help seal the housing 404 and prevent liquid infiltration into the housing 404. If the sensing device 400 includes an epoxy resin filler 108, then the housing 404 may not need side walls 409. The structure 470 projects from the outer surface 402 of the printed circuit board 411. In some versions, the protrusion 470 has a hemispherical shape (also called a dome) or semi-ellipsoidal shape. The protrusion 470 creates a well-defined smooth surface around the pressure-sensing element 420 and protects the pressure-sensing element 420. The protrusion 470 also increases the applanated area of the blood vessel 125. The protrusion 470 is made of a compressible elastomer polymer 471 that transmits pressure waves from the environment to the pressure-sensing elements 420. The polymer 471 may be a soft/flexible polymer. For example, the protrusion 470 may be made of polymerized siloxane (also called silicone, e.g., polydimethylsiloxane (PDMS)). Alternatively, protrusion 470 may be filled with silicone oil. FIG. 4C illustrates the sensing device 400 and pressure-sensing element 420 in FIG. 4A implanted against a blood vessel 125. Like the structures in FIGS. 2A and 3A, the protrusion 470 increases contact surface area between the pressure pressure-sensing element 420 and the wall of the blood vessel 126. Additionally, like the pressure-sensing device 100, the pressure-sensing device 400 may be coated with the coating 150 (including the alternating layers of Parylene 152 and SiOx 151) and the silicone gel 153.

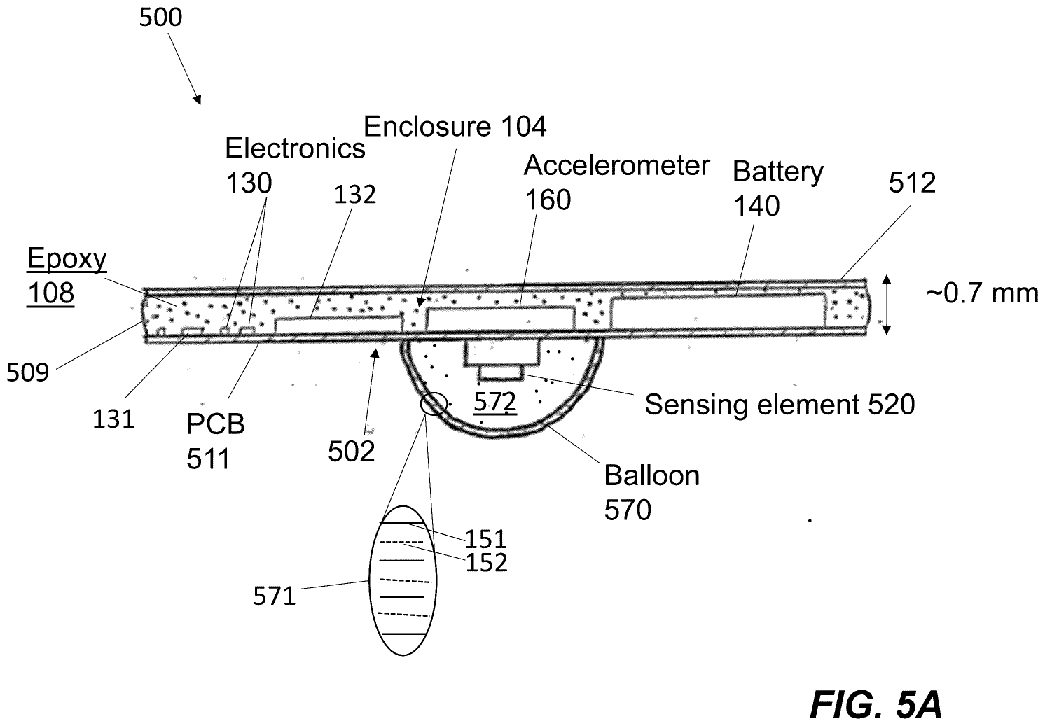

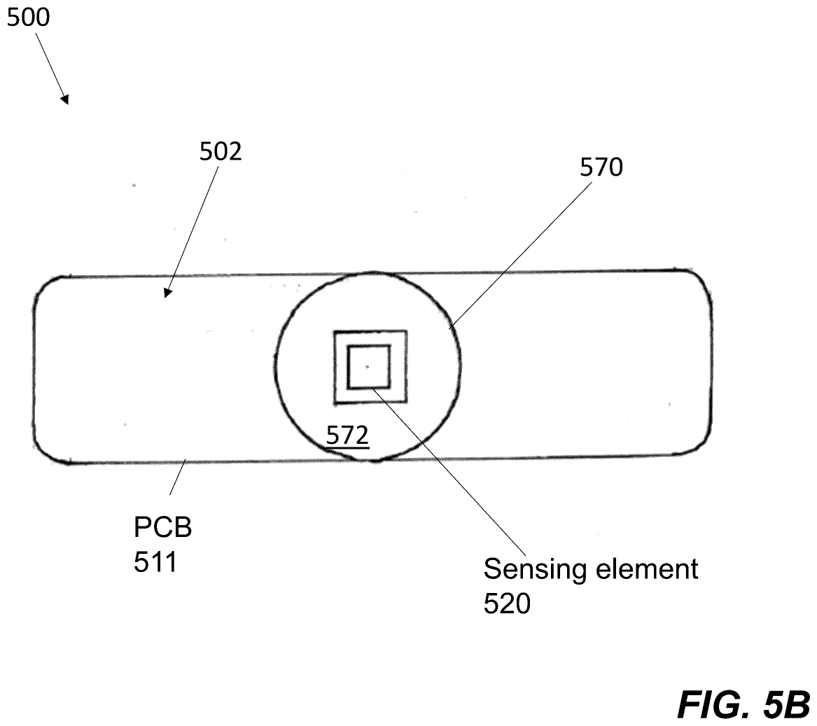

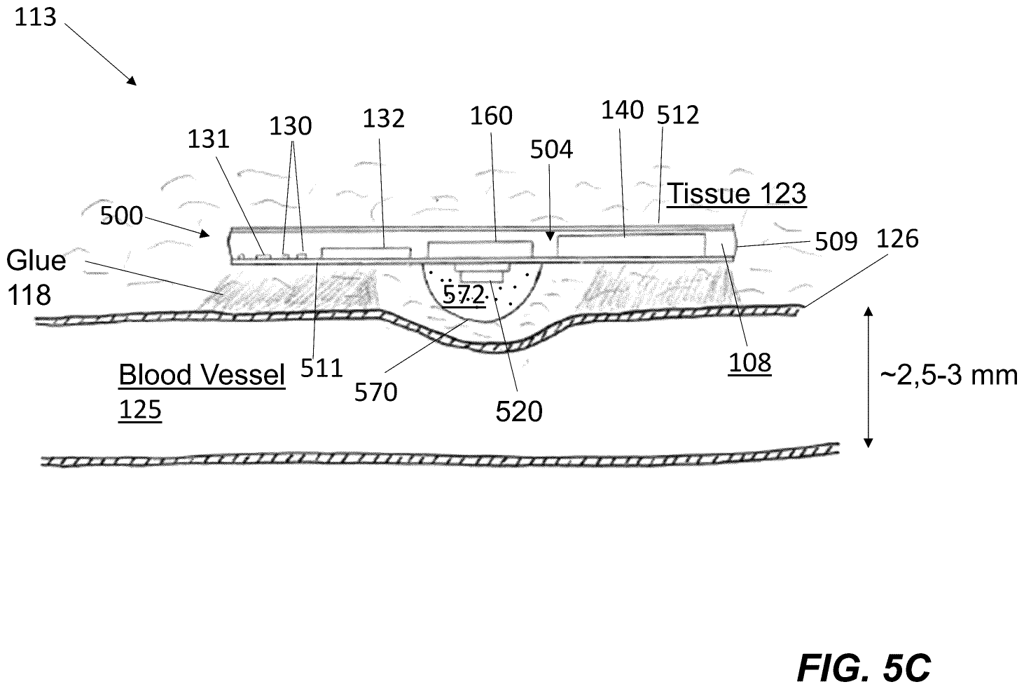

FIG. 5A illustrates a cross-section of an implantable sensing device 500 with a hemispherical or ovoid balloon structure 570 filled with liquid 572 disposed around the pressure-sensing element 520. FIG. 5B illustrates a plan view of the sensing device 500 in FIG. 5A. One of the substrates of the sensing device 500 is a printed circuit board (PCB) 511 and the other substrate of the sensing device 500 is a cover 512. The PCB 511 and cover 512 may be sandwiched together to form an enclosure or housing 504, as described above. The housing 504 may be at least partially filled with an inert polymer filler 108 (e.g., epoxy resin) to add mechanical rigidity to the pressure-sensing device 500 and to help seal the housing 504 and prevent liquid infiltration into the housing 504. If the sensing device 500 includes an epoxy resin filler 108, then the housing 504 may not need side walls 509. The balloon structure 570 projects from the outer surface 502 of the printed circuit board 511. FIG. 5C illustrates the sensing device 500 and the pressure-sensing element 520 in FIG. 5A implanted against a blood vessel 125. The balloon 570 may have a diameter similar to or slightly bigger than (e.g., about 150%) that of the blood vessel 125. The balloon 570 is made of a flexible and biocompatible polymer 571 (e.g., silicone or thermoplastic polyurethane) that is thin enough to transmit pressure waves and strong enough to resist puncture or leakage. As examples, the balloon 570 may be made of the coating material 150 (e.g., alternating layers of silicon dioxide 151 and Parylene-C 152). Instead of, or in addition to, Parylene-C, Parylene-F (a fluorinated poly(para-xylylene) polymer) may be used to form the balloon 570. Alternatively, the balloon 570 may be filled with a silicone gel (e.g., polydimethylsiloxane (PDMS)) or a silicone oil. Additionally, like the pressure-sensing device 100, the pressure-sensing device 500 may be coated with the coating 150 (including the alternating layers of Parylene 152 and SiOx 151) and the silicone gel 153.

The liquid 572 filling the balloon is an incompressible liquid. The force exerted by blood pressure is transmitted through the balloon 570 and the liquid 572 filling to the sensing element 520. The liquid 572 is inert or substantially inert so that it does not react with the pressure-sensing element 520 and is biocompatible in case of accidental leakage. As one example, the liquid 572 may be water, contrast fluid, saline, or Ringer's solution. As another example, the liquid 572 may be a hydrogel (e.g., a polyacrylate, a polymethacrylate, a polyurethane, a polyether, a polyester, a polyvinyl compound, a polycarbonate, or an epoxide). Alternatively, the balloon structure 570 may be filled with a gel or another fluid-like substance.

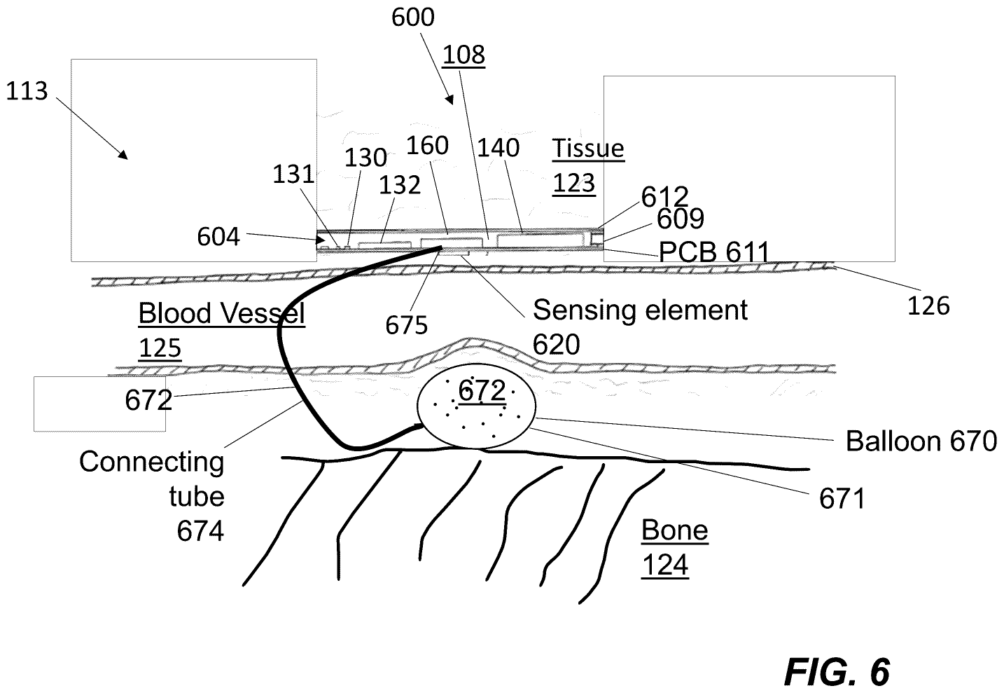

FIG. 6 illustrates a cross-section of an implantable pressure-sensing device 600 with a balloon 670 filled with liquid 672 fluidically coupled to the sensing element 620 with a connecting tube 674. One of the substrates of the sensing device 600 is a printed circuit board (PCB) 611 and the other substrate of the sensing device 600 is a cover 612. The PCB 611 and cover 612 may be sandwiched together to form an enclosure or housing 604, as described above. The housing 604 may be at least partially filled with an inert polymer filler 108 (e.g., epoxy resin) to add mechanical rigidity to the pressure-sensing device 600 and to help seal the housing 604 and prevent liquid infiltration into the housing 604. If the sensing device 600 includes an epoxy resin filler 108, then the housing 604 may not need side walls 609.

After being sterilized, the sensing device 600 is implanted so that it is disposed on or adjacent to a blood vessel wall 126 and the balloon 670 is positioned on a part of the blood vessel wall 126 opposite or approximately opposite the pressure-sensing device 600 between the blood vessel 125 and bone 124. In other words, the balloon 670 can be placed in between the blood vessel 125 and the bone 124. The balloon 670 may have a diameter similar to or slightly bigger than (e.g., about 150%) that of the blood vessel 125. The tube 674 connecting the balloon 670 to the pressure-sensing element 620 may have a length of about 200% to about 400% the diameter of the blood vessel 125. The balloon 670 may act in a fashion similar to a stethoscope. In other words, the blood pressure wave is transferred from the balloon 670, through the small tube 674 filled with fluid 672, to the pressure-sensing element 620. The balloon 670 is made of a flexible and biocompatible polymer 671 that is thin enough to transmit pressure waves and strong enough to resist puncture or leakage.

The liquid 672 filling the balloon is an incompressible and biocompatible liquid. The liquid 672 should be sterilized. As one example, the liquid 672 may be water, contrast fluid, saline, or Ringer's solution. As another example, the liquid 672 may be a hydrogel (e.g., a polyacrylate, a polymethacrylate, a polyurethane, a polyether, a polyester, a polyvinyl compound, a polycarbonate, or an epoxide). Alternatively, the balloon structure 670 may be filled with a gel or another fluid-like substance.

The tube 673 connecting the balloon 670 to the pressure-sensing element 620 may be made of the same material as the balloon and may also be filled with the same liquid filler 672. The tube 673 may be soldered, coupled, or otherwise attached to the balloon 670 on one end and onto a cavity 675 (like that shown in FIG. 6A) encompassing the pressure-sensing element 620 on the other end. The force exerted by blood pressure is transmitted through the balloon 670 and the liquid filling 672, through the connecting tube 674 filled with the liquid filling 661, and to the sensing element 620. The stethoscope configuration can be used to amplify the signal (like a typical stethoscope) and hence improve the robustness of the pressure signal and simplify discrimination from movement artifacts. Additionally, like the pressure-sensing device 100, the pressure-sensing device 600 may be coated with the coating 150 (including the alternating layers of Parylene 152 and SiOx 151) and the silicone gel 153.

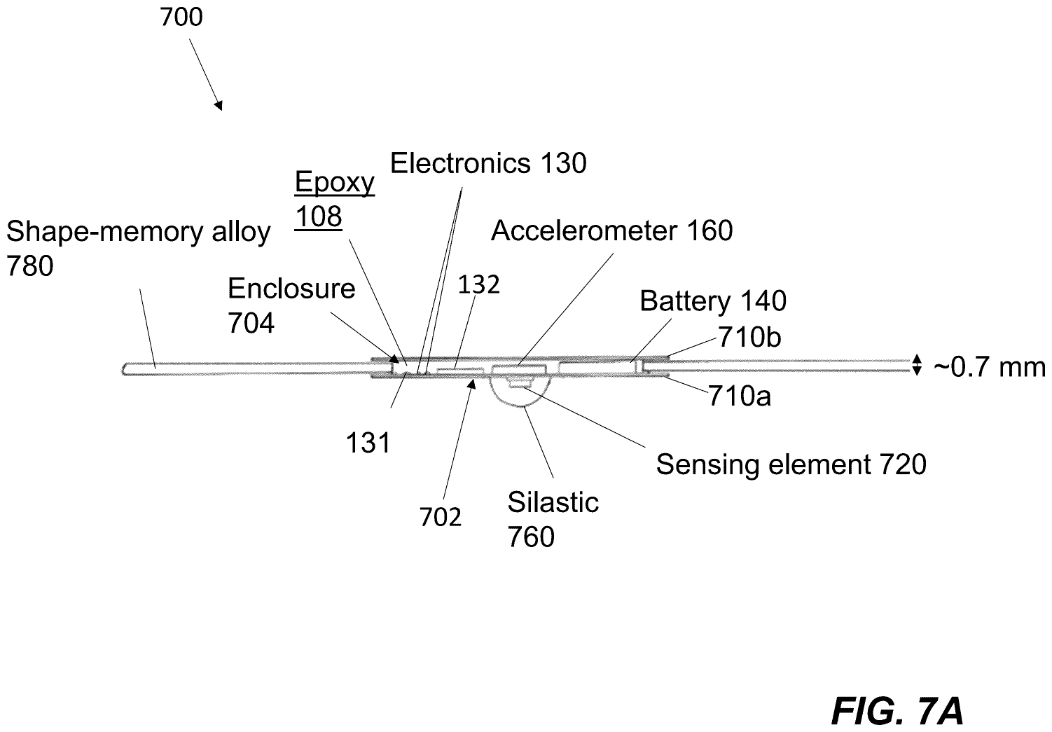

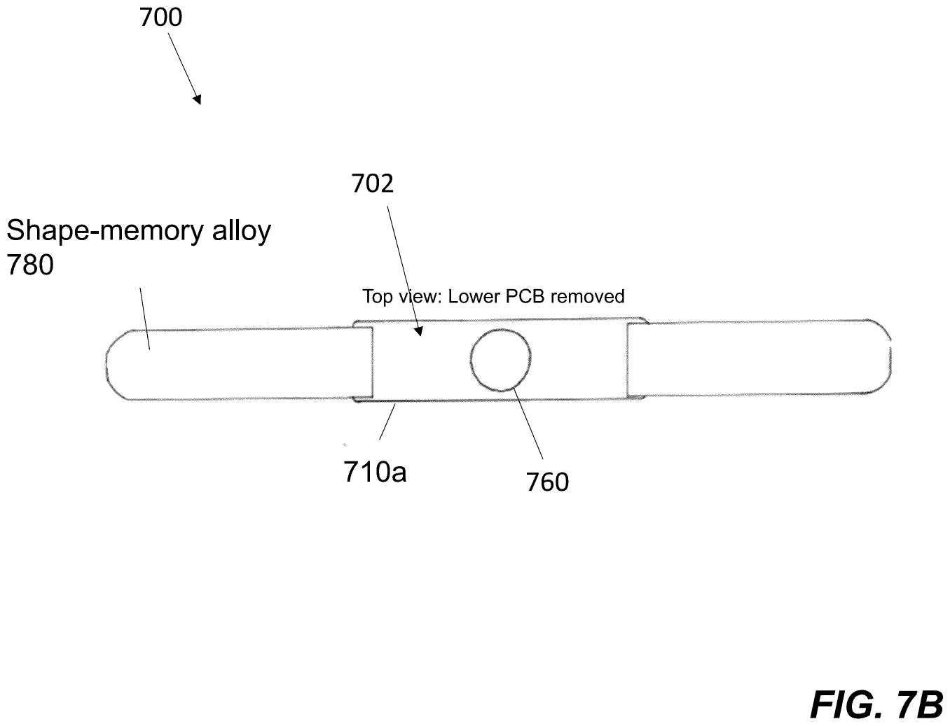

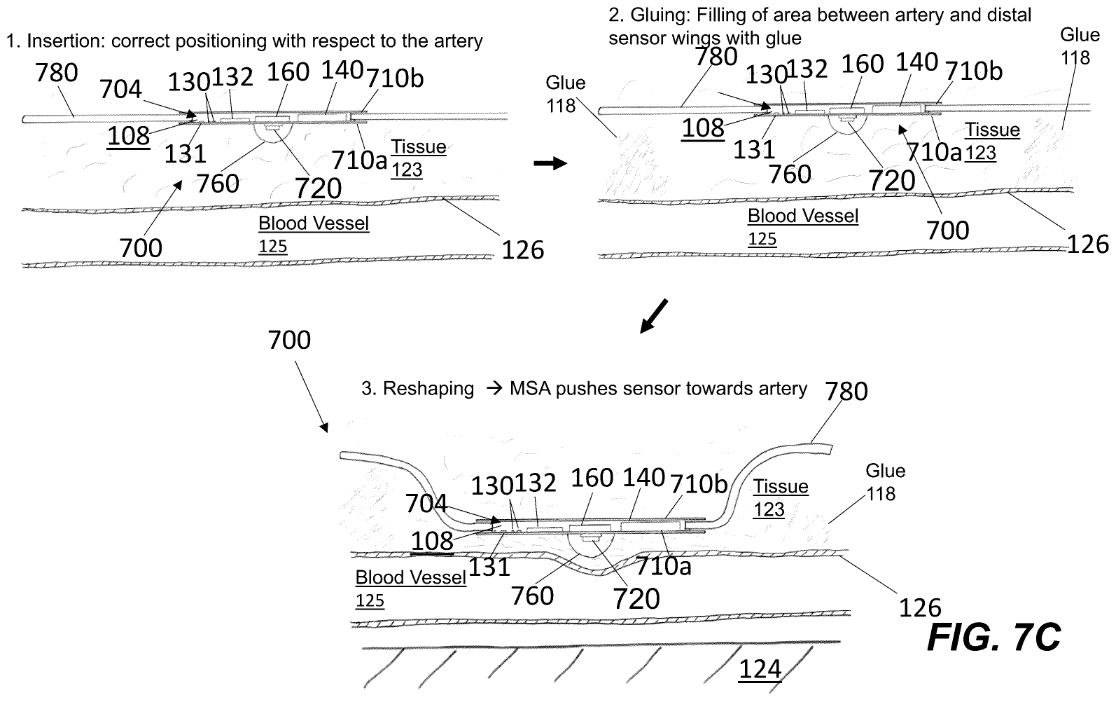

FIG. 7A illustrates a cross-section of an implantable sensing device 700 with shape-memory alloy (SMA) wings 780. FIG. 7B illustrates a plan view of the sensing device 700 in FIG. 7A. The SMA wings 780 help hold the sensing device 700 and the pressure-sensing element 720 in place once implanted. The sensing device 700 in FIG. 7A includes SMA wings 780 and a polymer elastomer protrusion 760 disposed on the pressure-sensing element 720 like in FIG. 4A. The SMA wings 780 may also be paired with the sensing device 700 without a structure on the sensing element 720, as in FIG. 1A, with a structure 270, 370, or 470 as in FIGS. 2A and 3A, or with a balloon 570 or 670 filled with liquid, gel, or another fluid-like substance as in FIGS. 5A and 6. As an example, the SMA 780 may be made of a nickel-titanium alloy (e.g., Nitinol). Each of the SMA wings 780 have a similar width as the housing, a length of about 100% to about 200% the length of the housing 704, and a width of about 0.5 mm to about 2 mm. Because of their shape, the SMA wings 780 push the pressure-sensing element 720 closer to the blood vessel 125 when deployed. FIG. 7A shows the ends of the SMA wings 780 partially inserted between the substrates 710a and 710b forming the sensing device housing 704 so that the SMA wings are held in place between the two substrates 710a and 710b. One of the substrates 710a of the sensing device 700 may be a printed circuit board (PCB) and the other substrate 710b of the sensing device 700 may be a cover 712. The housing 704 may be at least partially filled with an inert polymer filler 108 (e.g., epoxy resin) to add mechanical rigidity to the pressure-sensing device 700 and to help seal the housing 704 and prevent liquid infiltration into the housing 704. In other versions, the SMA wings 780 may be adhered to an outer surface 702 of the housing 704. Additionally, like the pressure-sensing device 100, the pressure-sensing device 700 may be coated with the coating 150 (including the alternating layers of Parylene 152 and SiOx 151) and the silicone gel 153.

FIG. 7C illustrates deployment of the sensing device 700 in FIG. 7A against a blood vessel 125 in vivo. The sensing device 700 is assembled with the SMA wings 780 having a planar shape. The sensing device 700 is implanted so that it is disposed adjacent to a blood vessel 125 and portions of the SMA wings 780 are adhered to the wall 126 of the blood vessel 125 with bio-glue 118 or suture technique. Once the sensing device 700 is fixed the SMA wings 780 are reshaped by being activated. The wings 780 may be activated with heat by a change of temperature. For example, the wings 780 may be kept at room temperature or cooler before implantation and may be activated by the heat of the body, since body temperature is higher than room temperature. The wings 780 may also be activated by mechanical activation. For example, the wings 780 may be pre-loaded on an applicator in their temporary constrained shape and then activated upon release from the applicator to reshape into a more stable form. Reshaping the SMA wings 780 causes the pressure-sensing element 720 to applanate the blood vessel wall 126 for tonometry.

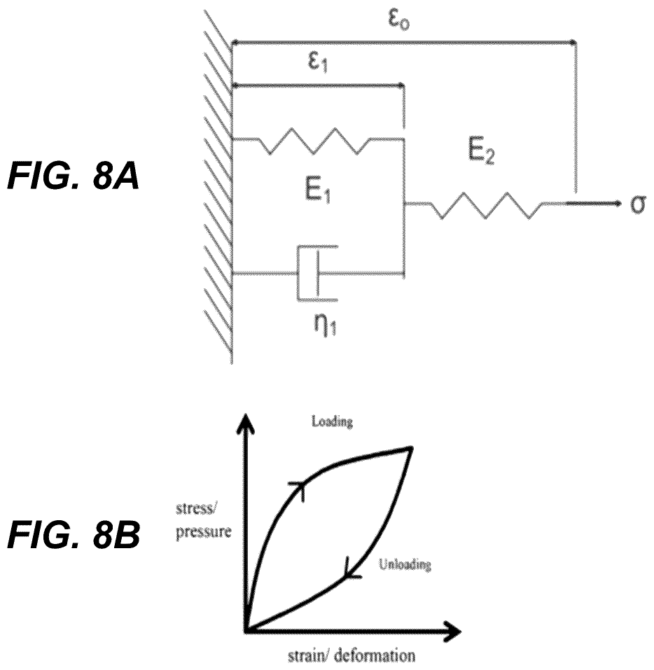

FIG. 8A is a viscoelastic model of the biomechanical properties of an artery, specifically the viscosity and the elasticity of a blood vessel wall 126. Epsilon1 (ε₁) represents the strain of the viscoelastic element and Epsilon0 (ε₀) represents the strain of the elastic element. E₁ and E₂ represent the elastic constants of the springs. Etal (η₁) represents the viscous constant. These parameters can be estimated from measurements and then incorporated into the biomechanical transfer function. The model in FIG. 8A is used to characterize the dynamic mechanical behavior of the blood vessel 125 for tonometry. These biomechanical properties are patient-specific and blood vessel-specific. The derived biomechanical properties are incorporated into the transfer function and used to determine blood pressure.

The biomechanical properties of a tissue or dome material determine how it responds and deforms when placed under stress for applanation tonometry. The model is a Kelvin-Voigt model with an additional spring. The Kelvin-Voigt model is a simple model viscoelastic material with a purely viscous damper and a purely elastic spring connected in parallel as shown in FIG. 8A. The Kelvin-Voigt model represents time-dependent viscous resistance to an applied force. The additional spring represents purely elastic behavior. When a force is applied to a blood vessel wall 126 for applanation tonometry, the blood vessel wall 126 demonstrates some instantaneous purely elastic deformation, represented by the additional spring. Upon additional application of force, the blood vessel wall 126 undergoes progressive viscoelastic deformation, represented by the modified Kelvin-Voigt model in FIG. 8A.

FIG. 8B is a plot of stress/pressure vs. strain/deformation for loading and unloading the pressure used to applanate the blood vessel wall 126. The loading pressure is higher than the unloading pressure and this difference in pressure is called hysteresis. The hysteresis represents the viscoelastic nature of the blood vessel wall.

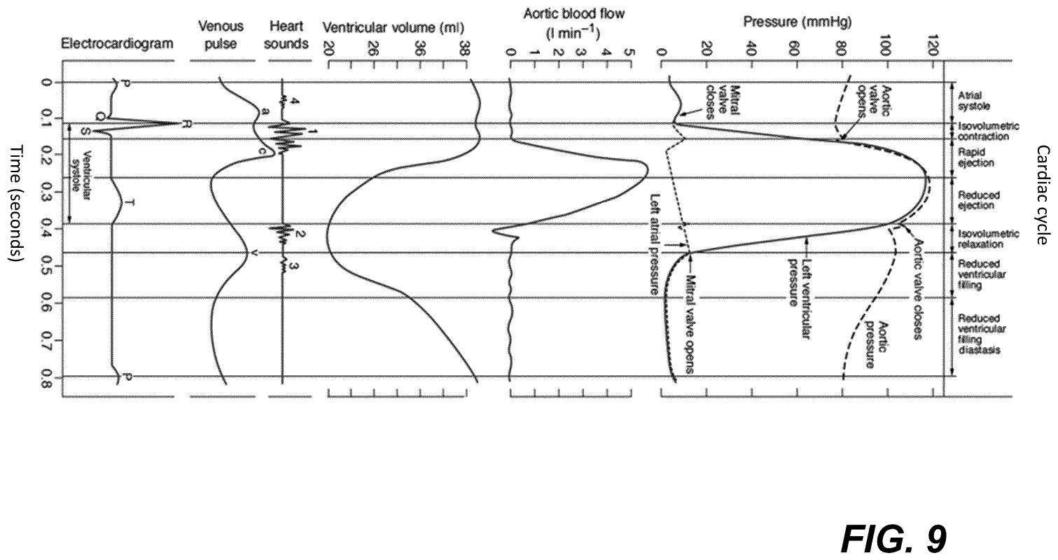

FIG. 9 is graph of the central pressure waveform over the cardiac cycle. FIG. 9 shows the pressure, aortic blood flow, ventricular volume, heart sounds, venous pulse, and electrocardiogram over the cardiac cycle in seconds. Central cardiac waveforms can be extrapolated from the peripheral radial artery waveform data measured with the sensing device. By measuring the pressure waveform, the pressure-sensing device can determine many parameters of the cardiac system, including heart rate, blood pressure, and other cardiac physiologic parameters. The pressure sensor measures waveform pressure through the cardiac cycle. The pressure waveform may also provide additional parameters of cardiovascular health including: central blood pressure (i.e., pressure of blood at the root of the aorta in the heart, which may be a predictor of subclinical cardiovascular disease); central pulse pressure (i.e., the pressure experienced by major organs like the heart, brain, and kidneys, which may be used to identify the risk of damage to major organs); augmentation pressure (i.e., a marker of the stiffness of an artery, which may be used to identify cardiovascular risk for patients with an elevated augmentation pressure); augmentation index (i.e., the burden stiff arteries place on the heart); subendocardial viability ratio, which may provide insight into how well an individual's heart can handle the stress of exercise; and heart rate, which provides insight into cardiovascular health.

Implantation of the pressure-sensing device can be done by open incision and placement under direct vision and alternatively can be done with a percutaneous or minimally invasive surgery. For example, percutaneous implantation may use a balloon catheter to dilate the subcutaneous space, a needle introducer to implant the sensing device, and a guide wire to position the sensing device accurately. The person performing this procedure may use ultrasound imaging to visualize the radial artery, the balloon catheter, the needle, the guide wire, and the sensing device during the procedure to position the sensing device accurately. The person performing the procedure may also use conventional blood pressure sensors to compare to the implanted pressure-sensing device's measurements to ascertain that the implanted pressure-sensing device is positioned accurately for measuring blood pressure.

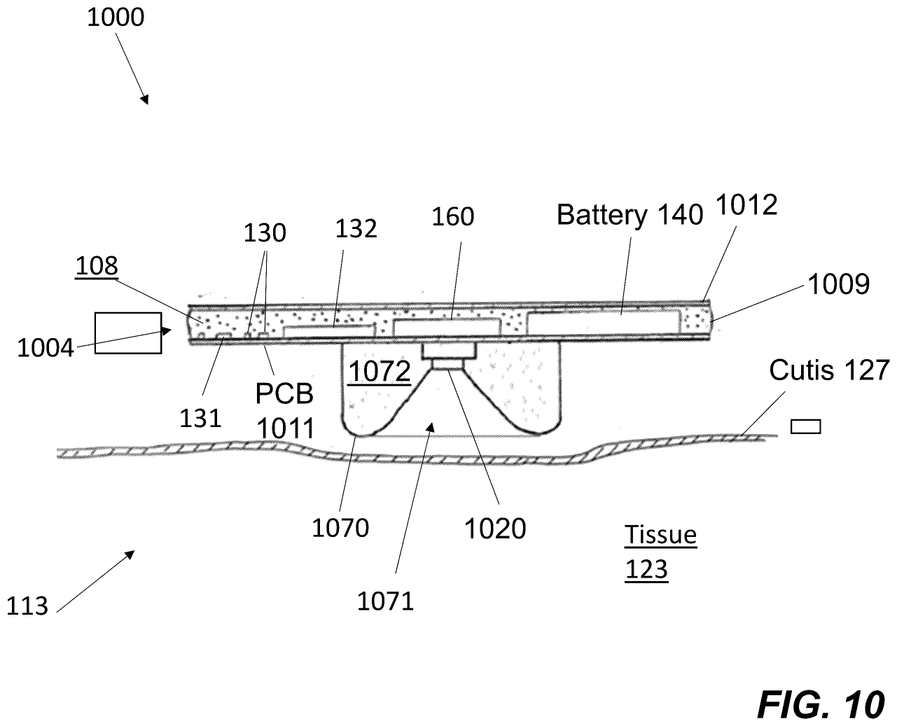

FIG. 10 illustrates a cross-section of a sensing device 1000 that may be used externally. The sensing device 1000 is placed on the cutis 127 or outer skin of a mammalian patient to measure the patient's BP using applanation tonometry. It can be held in place by hand, with a cuff or bandage, or even with a temporary adhesive. One of the substrates of the sensing device 1000 is a printed circuit board (PCB) 1011 and the other substrate of the sensing device 1000 is a cover 1012. The PCB 1011 and cover 1012 may be sandwiched together to form an enclosure or housing 1004, as described above. The housing 1004 may be at least partially filled with an inert polymer filler 108 (e.g., epoxy resin) to add mechanical rigidity to the pressure-sensing device 1000 and to help seal the housing 1004 and prevent liquid infiltration into the housing 1004. If the sensing device 1000 includes an epoxy resin filler 108, then the housing 1004 may not need side walls 1009. The structure's concavity 1071 is centered around and surrounds the pressure-sensing element 1020 to focus the pulse wave onto the pressure-sensing element 1020. While FIG. 10 illustrates a sensing device 1000 with conical structure 1070 to focus the pulse wave onto the pressure-sensing device's 1000 pressure-sensing element 1020, any of the sensing devices described above, including with or without any of the focusing structures (e.g., 270, 370, 470, 570, or 670), can be used for measuring the patient's BP externally (i.e., without implanting the sensing device inside of the patient). The conical structure 1070 may be made of an inert biocompatible polymer 1072 as described above. Some versions of the external sensing device 1000 include an internal battery 140 that provides power for operating the sensing device. Additionally, like the pressure-sensing device 100, the pressure-sensing device 1000 may be coated with the coating 150 (including the alternating layers of Parylene 152 and SiOx 151) and the silicone gel 153.

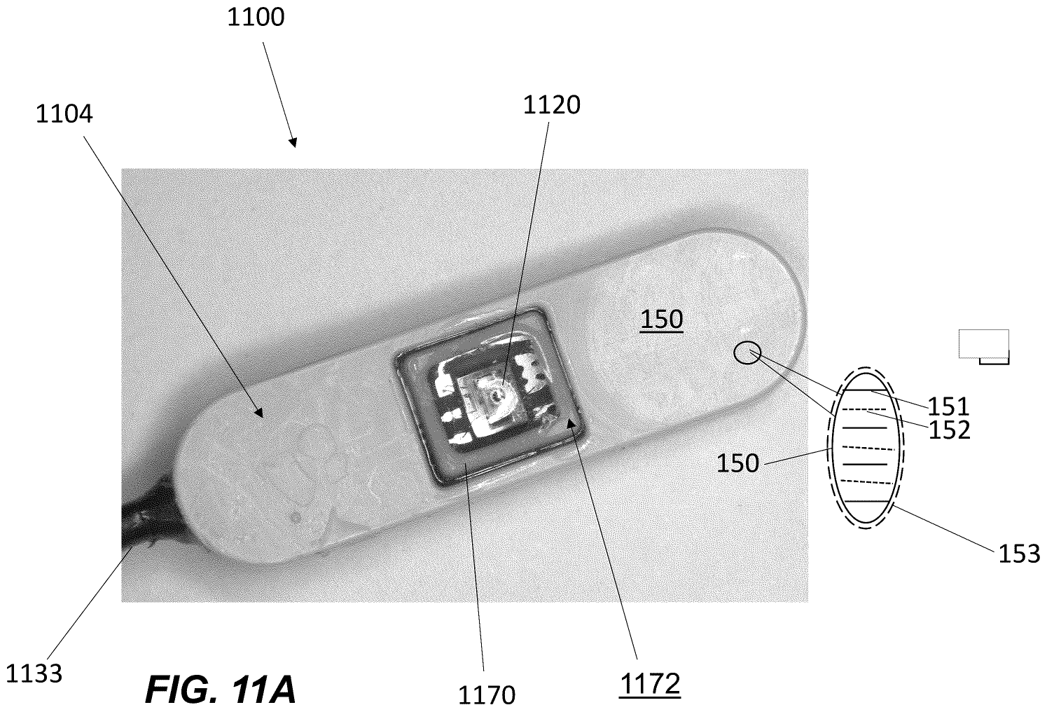

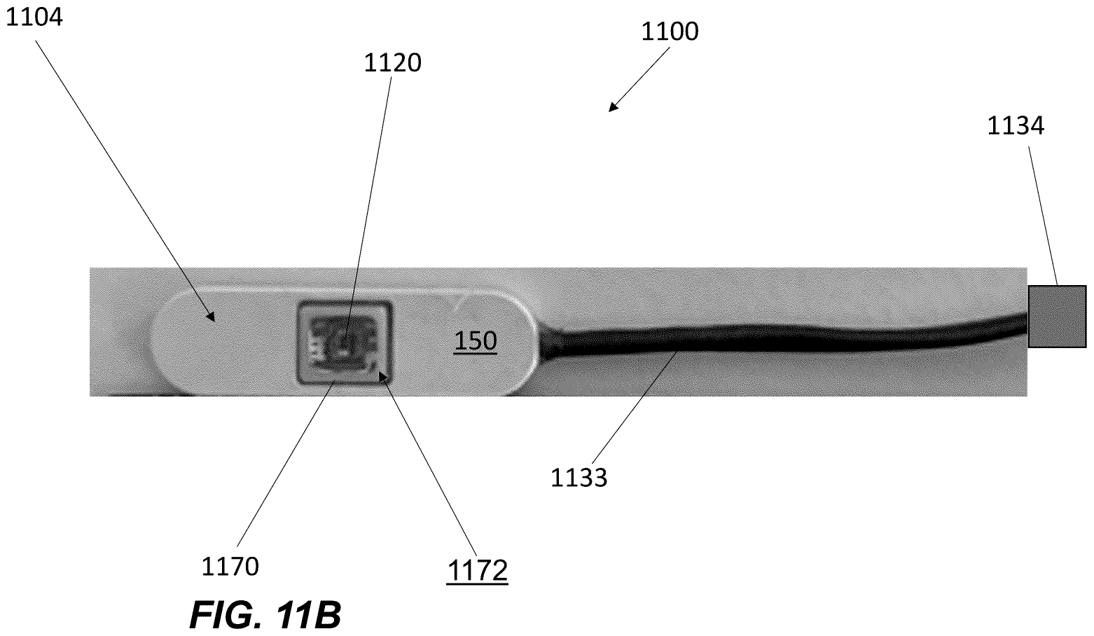

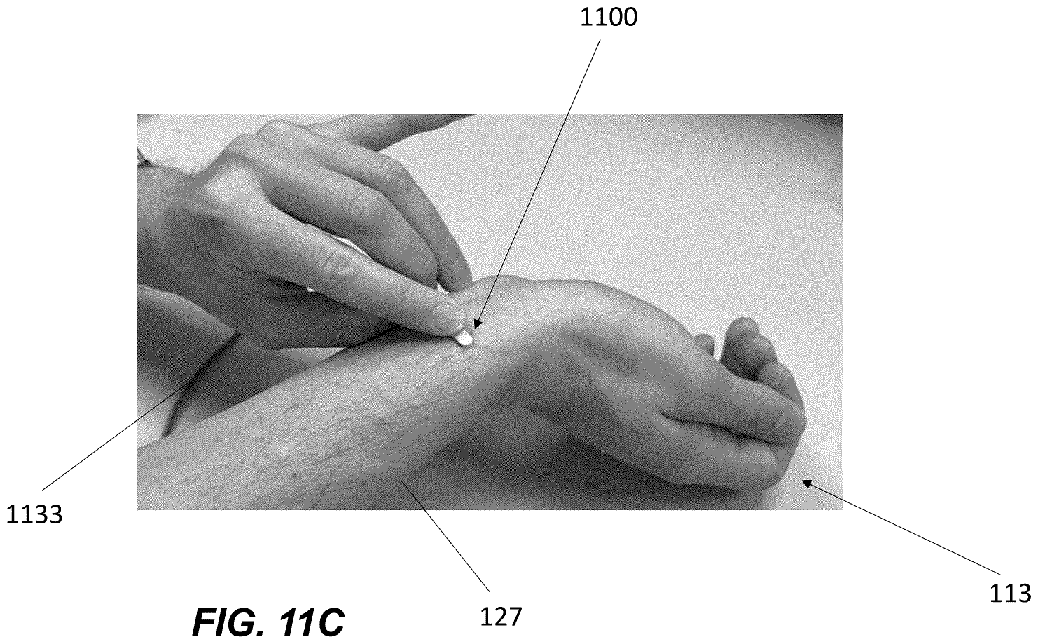

FIGS. 11A-11C show photos of a sensing device 1100 that may be used externally. FIGS. 11A and 11B are plan view photos of the sensing device 1000. The sensing device 1100 includes a pressure-sensing element 1120. The pressure-sensing device 1100 may also include a focusing structure 1170. The focusing structure 1170 is centered around and surrounds the pressure-sensing element 1120 to focus the pulse wave onto the pressure-sensing element 1020. The conical structure 1170 may be made of an inert biocompatible polymer 1172 as described above. Any of the sensing devices described above, including with or without any of the focusing structures (e.g., 270, 370, 470, 570, or 670), can be used for measuring the patient's BP externally (i.e., without implanting the sensing device inside of the patient). Additionally, like the pressure-sensing device 100, the pressure-sensing device 1100 may be coated with the coating 150 (including the alternating layers of Parylene 152 and SiOx 151) and the silicone gel 153.

The sensing device 1100 may also include a substrate (e.g., a PCB) and a cover beneath the coating 150. The PCB and cover may be sandwiched together to form an enclosure or housing 1104, as described above. The housing 1104 may be at least partially filled with an inert polymer filler (e.g., epoxy resin) as discussed above. The housing 1104 may also include various electronics including an accelerometer, controller, and antenna as described and illustrated above. In one embodiment, the sensing device 1100 may not include an internal battery and instead can be electrically coupled to an external power source 1134 via conductive links (e.g., a plug and/or a wire 1033) as shown in FIG. 11B.

FIG. 11C shows the sensing device 1000 being used externally. Like the sensing device of FIG. 10, the sensing device 1100 is placed on the cutis 127 or outer skin of a mammalian patient to measure the patient's BP using applanation tonometry. It can be held in place by hand, with a cuff or bandage, or even with a temporary adhesive.

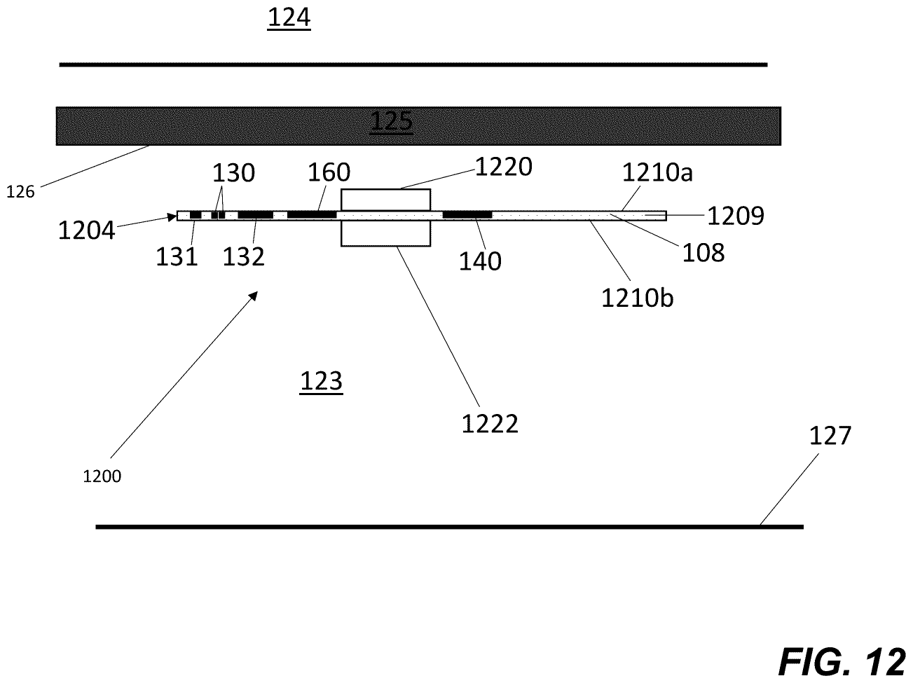

FIG. 12 illustrates an implantable pressure-sensing device 1200 with two pressure sensors 1220 and 1220 (e.g., a first pressure-sensing element 1220 as described above and a second pressure-sensing element 1222). The first pressure-sensing element 1220 may be positioned on the proximal side of the sensing device 1200 (i.e., the side of the blood vessel 125 and facing the blood vessel 125). The second pressure-sensing element 1222 may be placed on the distal side of the sensing device 1200 (i.e., the side opposite the blood vessel 125). The second pressure-sensing element 1222 may also be configured to detect changes in pressure distribution in the body. For example, the second pressure-sensing element may measure tissue pressure from the surrounding tissue 123. Tissue pressure may be affected by changes in fluid distribution in the mammalian body due to motion and/or position of the body. The second pressure-sensing element 1222 may also be a reference sensor. The second pressure-sensing element 1222 may be of the same type and made of the same materials as the first pressure-sensing element 1220. Alternatively, the second pressure-sensing element 1222 may be a different type of pressure-sensing element and/or may be made of any different suitable materials.

Some versions of the sensing device 1200 include two substrates 1210a and 1210b sandwiched together to form an enclosure or housing 1204, as described above. One of the substrates 1210a of the sensing device 1200 may be a printed circuit board (PCB). Another substrate 1210b may be a cover. The housing 1204 may be at least partially filled with an inert polymer filler 108 (e.g., epoxy resin) to add mechanical rigidity to the pressure-sensing device 1200 and to help seal the housing 1204 and prevent liquid infiltration into the housing 1204. If the sensing device 1200 includes an epoxy resin filler 108, then the housing 1204 may not need side walls 1209. Additionally, like the pressure-sensing device 100, the pressure-sensing device 1200 may be coated with the coating 150 (including the alternating layers of Parylene 152 and SiOx 151) and the silicone gel 153.

In another embodiment, both the first pressure-sensing element 1220 and the second pressure-sensing element 1222 may be located on the same substrate (e.g., a PCB). For example, the first pressure-sensing element 1220 may be positioned on one side of substrate 1210a (e.g., the proximate size) and the second pressure-sensing element 1222 may be positioned on the other side of substrate 1210a (e.g., the distal side). In this embodiment, the sensing device 1200 may include only one substrate (e.g., a PCB). The first pressure-sensing element 1220 and/or the second pressure-sensing element 1222 may be at least partially exposed through the coating 150 (e.g., by a hole in the coating, for example).

After being sterilized, the sensing device 1200 is implanted so that it is disposed on or adjacent to a blood vessel wall 126 with the first sensing element 1220 facing the blood vessel 125. The sensing device 1200 may be used with or without any of the focusing structures described above (e.g., 270, 370, 470, 570, 670, or 1070). The sensing device 1200 can also be used for measuring the patient's BP externally (i.e., without implanting the sensing device 1200 inside of the patient). The sensing device 1200 may also be used with the SMA wings and/or polymer elastomer protrusion described above in FIGS. 7A-7C.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An implantable pressure-sensing device comprising:
a substrate configured to secure the implantable pressure-sensing device within 0 mm to 10 mm from a blood vessel;
a pressure-sensing element projecting from an outer surface of the substrate and configured to sense blood pressure within the blood vessel;
circuitry disposed on the substrate and in electrical communication with the pressure-sensing element and configured to receive data from the pressure-sensing element; and
a power management system disposed on the substrate and configured to provide power to the pressure-sensing element and the circuitry.

2. The implantable pressure-sensing device of claim 1, further comprising:
a coating disposed on the substrate and the pressure-sensing element and hermetically sealing the substrate and the pressure-sensing element, the coating including layers of ceramic and polymer.

3. The implantable pressure-sensing device of claim 2, wherein the coating has a thickness of about 1 μm to about 1 mm.

4. The implantable pressure-sensing device of claim 2, wherein the coating comprises 3 layers to 14 alternating layers of SiOx and Parylene.

5. The implantable pressure-sensing device of claim 1, further comprising:
an accelerometer disposed on the substrate and configured to detect body movements.

6. The implantable pressure-sensing device of claim 1, wherein the pressure-sensing element is a first pressure-sensing element and further comprising:
a second pressure-sensing element disposed on the substrate and configured to detect changes in pressure distribution in a mammalian body.

7. The implantable pressure-sensing device of claim 1, further comprising:
a structure disposed around the pressure-sensing element and projecting from the substrate and configured to transmit pressure waves from the blood vessel towards the pressure-sensing element.

8. The implantable pressure-sensing device of claim 7, wherein the structure has a dome shape centered around the pressure-sensing element.

9. The implantable pressure-sensing device of claim 7, wherein the structure has a valley shape centered around the pressure-sensing element.

10. The implantable pressure-sensing device of claim 7, wherein the structure is shaped in a way to maximize a signal to noise ratio.

11. The implantable pressure-sensing device of claim 1, further comprising:
an elastomer disposed on the pressure-sensing element and configured to transmit pressure waves to the pressure-sensing element.

12. The implantable pressure-sensing device of claim 1, further comprising:
a first shape-memory alloy wing mechanically coupled to the substrate; and
a second shape-memory alloy wing mechanically coupled to the substrate opposite the first shape-memory alloy wing,
wherein the first shape-memory alloy wing and the second shape-memory alloy wing are configured to be deployed after the implantable pressure-sensing device is implanted to secure the implantable pressure-sensing device against the blood vessel.

13. The implantable pressure-sensing device of claim 1, wherein the substrate is a first substrate and further comprising:
a second substrate,
wherein:
the first substrate and the second substrate are part of a rigid housing with an enclosure between the first substrate and the second substrate;
the circuitry and the power management system are disposed in the enclosure; and
the enclosure is filled with an inert polymer filler.

14. The implantable pressure-sensing device of claim 13, wherein the inert polymer filler comprises epoxy resin.

15. The implantable pressure-sensing device of claim 1, wherein the implantable pressure-sensing device has one dimension that is about 10% to about 100% of a width of the blood vessel.

16. The implantable pressure-sensing device of claim 15, wherein the implantable pressure-sensing device has a second dimension that is about 25% to about 1000% of the width of the blood vessel.

17. The implantable pressure-sensing device of claim 15, wherein the blood vessel is selected from the group consisting of an artery, a vein, a capillary, or a graft.

18. The implantable pressure-sensing device of claim 17, wherein the blood vessel is selected from the group consisting of a radial artery, an ulnar artery, a brachial artery, or a sub-clavian artery.

19. The implantable pressure-sensing device of claim 15, wherein the blood vessel is a blood vessel in a lower limb.

20. The implantable pressure-sensing device of claim 15, wherein the blood vessel is a great vessel.

21. An implantable pressure-sensing device comprising:
a substrate configured to be secured within 0 mm to 10 mm of a blood vessel; and
a pressure-sensing element projecting from the substrate and configured to make measurements of blood pressure in the blood vessel when the substrate is secured within 0 mm to 10 mm of the blood vessel.
